(19) United States
(12) Patent Application Publication
(10) Pub. No.: US 2025/0099058 A1
(43) Pub. Date: Mar. 27, 2025

(54) SYSTEMS AND METHODS FOR ASSESSING AORTIC VALVE CALCIFICATION USING CONTRAST-ENHANCED COMPUTED TOMOGRAPHY (CT)

(71) Applicant: McMaster University, Hamilton (CA)

(72) Inventors: Zahra Keshavarz Motamed, Ancaster (CA); Mohamed Abdelkhalek, Hamilton (CA)

(21) Appl. No.: 18/730,936

(22) PCT Filed: Jan. 20, 2023
(86) PCT No.: PCT/CA2023/050057

§ 371 Date: Jul. 22, 2024

Related U.S. Application Data
(60) Provisional application No. 63/301,823, filed on Jan. 21, 2022.

(51) Int. Cl. A61B 6/50; A61B 6/00; A61B 6/03; G06T 5/20; G06T 5/70; G06T 7/00; G06T 7/11; G06T 7/62; G06T 7/66; G16H 30/40

(52) U.S. Cl. CPC A61B 6/503; A61B 6/032; A61B 6/5205; A61B 6/5217; G06T 5/20; G06T 5/70; G06T 7/0012; G06T 7/11; G06T 7/62; G06T 7/66; G16H 30/40; G06T 2207/10081; G06T 2207/20036; G06T 2207/30048; G06T 2207/30096

(57) ABSTRACT

Described is a method for assessing aortic valve calcification using contrast-enhanced CT. The method comprises: receiving CT images of the aortic valve; pre-processing received images to have a field of view focused on an aortic root corresponding to the aortic valve; implementing a fast-marching method for the pre-processed images to segment the aortic valve from surrounding tissue to generate an aortic root model; determining multiple principal axes and multiple landmark points based on the pre-processed images and the aortic root model to define a local coordinate system relative to leaflets of the aortic valve; generating a calcification model based on the pre-processed images and aortic root model by iteratively changing an initial estimate of calcific HU threshold until a minimum false positive rate FPR criterion is reached; and generating an indicator quantifying calcification of the aortic valve based on the calcification model, the principal axes and the landmark points.

### RELATED APPLICATIONS

This application claims the benefit of priority to U.S. Provisional Application No. 63/301,823 filed Jan. 21, 2022, the entire contents of which are hereby incorporated by reference.

### FIELD

The described embodiments relate to systems and methods for assessing aortic valve calcification, and more specifically relate to systems and methods for assessing aortic valve calcification using contrast-enhanced computed tomography (CT).

### BACKGROUND

Calcification in the cardiovascular system is associated with adverse outcomes [1-3]. Although originally considered a passive process that occurs naturally with aging, calcification development is, in fact, highly complex and influenced by several factors [1, 4, 5]. Different types of calcifications can be categorized depending on the region where they are found, namely aortic valve calcification (AVC), mitral valve calcification, coronary artery calcification and thoracic aortic calcification. Vascular calcification may be described as an increase in the thickness of arterial walls that eventually become calcified plaques that may obstruct blood flow and adversely affect wall stiffness [4, 5]. Cardiac valve calcification is of great importance in the context of characterizing valve stenosis [6] and has many implications for surgical intervention planning and post-operative assessment [7].

Aortic valve calcification (AVC) strongly influences native and artificial valve behavior and is a key feature involved in the development and progression of aortic stenosis (AS) [8]. AVC is presented in aortic sclerosis, a disease in which the valve leaflets begin to thicken and develop regions of focal calcification, and overtime can progress to AS [8, 9]. AS is characterized as a gradual decrease of the valve orifice area leading to left ventricular outflow tract (LVOT) obstruction and left ventricular (LV) hypertrophy due to an increased afterload [6].

Prior to the recent introduction of the minimally invasive transcatheter aortic valve replacement (TAVR), surgical aortic valve replacement was the only possible intervention for severe AS. As TAVR is becoming more frequent [10], the procedure is continuously advancing with improvements in valve and delivery system designs, as well as increased clinician experience [10]. It is therefore critical that during procedural planning, valuable prognostic information is collected to help plan and optimize patient-specific interventions [11]. Primarily, detailed calcification assessment must be included in this process [11, 12] as AVC is associated with a variety of peri-procedural complications [11, 13]. In the context of TAVR, the new valve may be restricted from expanding completely due to native calcification. The presence of leaflet and/or annular/LVOT calcification is a risk factor to paravalvular leakage surrounding the implant [14], annular rupture, aortic root injury or conduction abnormalities which may increase the risk of mortality [15, 16].

### SUMMARY

Simple modifications to the standard Agatston technique when using contrast-enhanced CT images have been previously investigated [17-22]. These approaches were mainly motivated by the idea of using a new cut-off thresholding value, either fixed or dynamically determined based on luminal attenuation. In this regard, the disclosed systems and methods do not explicitly rely on luminal attenuation. Instead, initial calcification regions may be determined based on the intensity characteristics of a bounding aortic root segmentation. Using those initial conditions, an iterative region growing method may adjust the Hounsfield band for calcific detection gradually, until satisfying certain overlap criteria between calcific and non-calcific segments in the image. The disclosed systems and methods provide a novel anatomically based regional mapping scheme that can provide a quantitative description of location, quantity, and average intensity of calcific deposition. The diagnostic abilities of the disclosed systems and methods can be demonstrated using the novel analyses and interpretations of example clinical data described herein. The disclosed systems and methods can provide a novel in-vivo quantitative description of aortic valve calcification using high resolution CT imaging.

The disclosed systems and methods may be used for assessing aortic valve calcification using contrast-enhanced CT for any health condition, for example, healthy or unhealthy conditions, symptomatic or asymptomatic conditions etc. The disclosed systems and methods may be used for assessing aortic valve calcification using contrast-enhanced CT for AS or any other aortic valve diseases (e.g., tricuspid aortic stenosis and bicuspid aortic stenosis, aortic valve regurgitation, paravalvular leakage after intervention/surgery). The assessment may be performed for both pre-intervention and post-intervention cases. The intervention can be any suitable medical procedure or surgery to address a health condition. For example, and without limitation, the intervention can be TAVR, and the disclosed systems and methods may be used for assessing aortic valve calcification both pre-TAVR and post-TAVR.

In one aspect, there is provided a method for assessing calcification of an aortic valve. The method comprises: receiving computed tomography (CT) images of the aortic valve; pre-processing the received images to have a field of view focused on an aortic root corresponding to the aortic valve; implementing a fast-marching method for the pre-processed images to segment the aortic valve from surrounding tissue to generate an aortic root model; determining multiple principal axes and multiple landmark points based on the pre-processed images and the aortic root model to define a local coordinate system relative to leaflets of the aortic valve; generating a calcification model based on the pre-processed images and the aortic root model by iteratively changing an initial estimate of calcific Hounsfield (HU) threshold until a minimum false positive rate (FPR) criterion is reached; and generating an indicator quantifying calcification of the aortic valve based on the calcification model, the principal axes and the landmark points.

In one or more embodiments, generating the indicator includes generating one or more maps quantifying calcification of the aortic valve.

In one or more embodiments, the method further comprises measuring one or more local and/or global geometric parameters quantifying calcification of the aortic valve based on the one or more maps.

In one or more embodiments, the geometric parameters include one or more of a physical volume, multiple principal orientation axes, a center of mass, multiple boundary points, a roundness, a flatness, an elongation, an equivalent spherical radius, equivalent ellipsoidal diameters and a fractal dimension index of each calcific lesion in the generated calcification model.

In one or more embodiments, the received images are pre-processed to have the field of view include an interface between the aortic valve and a left ventricular outflow tract (LVOT), and a part of the ascending aorta after a Sino-tubular junction (STJ); and to have the field of view exclude any other surrounding structures.

In one or more embodiments, generating the aortic root model includes performing morphological operations to obtain a smoothed surface.

In one or more embodiments, the multiple principal axes correspond to an anatomical short axis view, and two long axis views that are perpendicular to the “En-face” short axis view.

In one or more embodiments, the method further comprises generating an anatomical N region volume map and/or an anatomical N region average intensity map quantifying calcification of the aortic valve, the anatomical N region volume map and the anatomical N region average intensity map being based on the calcification model, the principal axes, a Sino-tubular junction (STJ) height, an annular radius, angles between leaflets of the aortic valve, wherein N defines a number of discrete regions in the volume map and/or the average intensity map.

In one or more embodiments, the one or more maps quantifying calcification include one or more of a regional calcification map, a radial distance map, a longitudinal distance map and a calcification intensity map.

In one or more embodiments, the received CT images are contrast-enhanced CT images.

In one or more embodiments, the method further comprises diagnosing, monitoring or prognosing aortic valve stenosis (AS) in a subject based on the one or more local and/or global geometric parameters.

In one or more embodiments, a procedural risk assessment and/or a complication/event prediction is conducted prior to a transcatheter aortic valve replacement (TAVR).

In another aspect, there is provided a system for assessing calcification of an aortic valve. The system comprises: a processor; and a memory storing processor-executable instructions, wherein the instruction configure the processor to perform any of the methods described herein.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF THE DRAWINGS

One or more embodiments will now be described in detail with reference to the drawings, in which:

FIGS. 1a-1d show visual representations of a computational framework for assessing aortic valve calcification, in accordance with one or more embodiments.

FIGS. 2a-2c show visual representations of an example calcification detection algorithm that may be used with the framework shown in FIG. 1.

FIGS. 3a-3h show example results of detected calcification in contrast versus non-contrast representative cases, using the framework shown in FIG. 1.

FIGS. 4a-4f show example statistics and data plots for parameters obtained using the framework shown in FIG. 1.

FIGS. 5a-5h show example patterns of leaflet calcification in AS for contrast representative cases using the framework shown in FIG. 1.

FIGS. 6a and 6b show examples of comparative analysis of calcific detection methods and regional distribution of calcification across the cohort (N=178).

FIG. 7a shows a method for assessing calcification of an aortic valve, in accordance with one or more embodiments.

FIG. 7b shows another method for assessing calcification of an aortic valve, in accordance with one or more embodiments.

FIG. 8 shows a system for assessing calcification of an aortic valve, in accordance with one or more embodiments.

FIG. 9 shows a device for assessing calcification of an aortic valve, in accordance with one or more embodiments.

FIG. 10 shows example visual representations of the described computational framework for the assessment of calcification pattern and structure in contrast enhanced CT images for TAVR framework, in accordance with one or more embodiments.

FIG. 11 shows examples of total and regional calcification in pre vs post TAVR in representative cases, in accordance with one or more embodiments.

FIG. 12 shows examples of landing zone calcific distribution in post-procedural complications, in accordance with one or more embodiments.

FIG. 13 shows example landing zone calcific distribution in peri-procedural events, in accordance with one or more embodiments.

FIG. 14 shows example classification power analysis of regional landing zone calcification maps using binomial logistic regression analysis, in accordance with one or more embodiments.

### DESCRIPTION OF EXEMPLARY EMBODIMENTS

### Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example”.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers or computing devices may be a server, network appliance, embedded device, computer expansion module, a personal computer, laptop, personal data assistant, cellular telephone, smart-phone device, tablet computer, a wireless device or any other computing device capable of being configured to carry out the methods described herein.

In one embodiment, the communication interface may be a network communication interface. In embodiments in which elements are combined, the communication interface may be a software communication interface, such as those for inter-process communication (IPC). In still other embodiments, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

### EXAMPLES

The following non-limiting examples are illustrative of the present disclosure.

### Methods

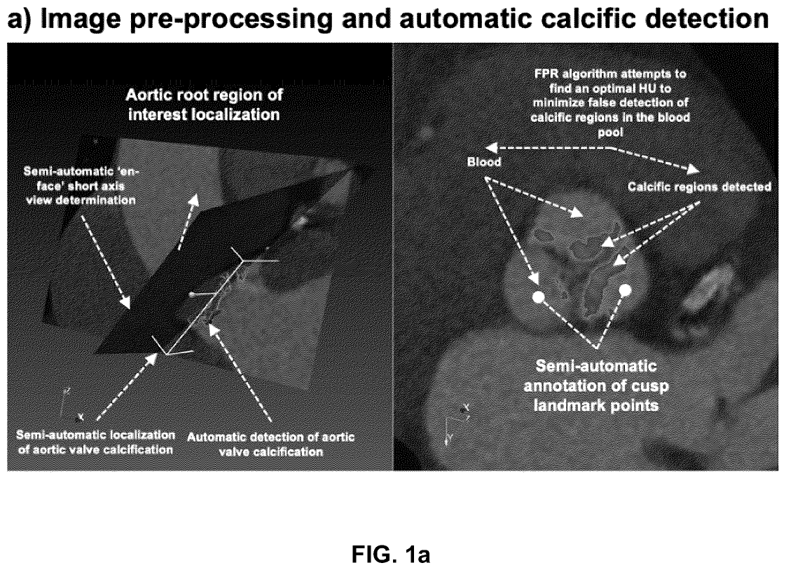

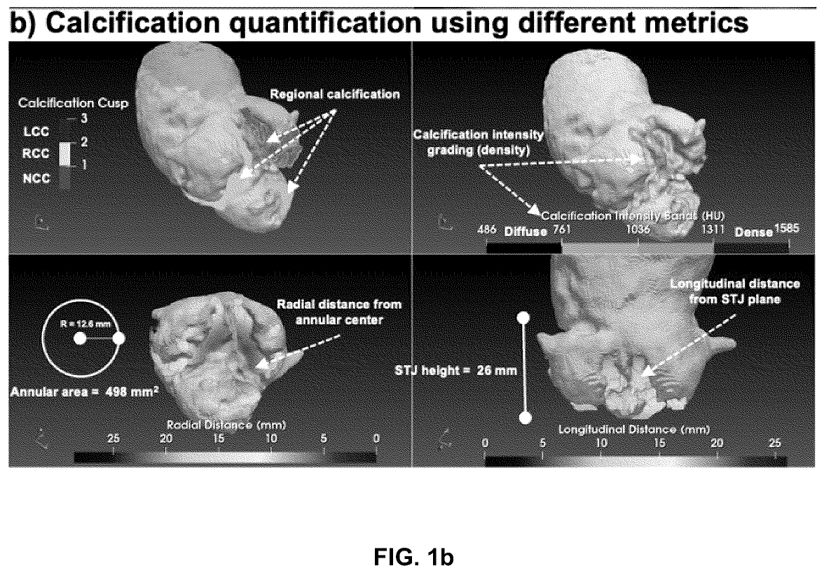

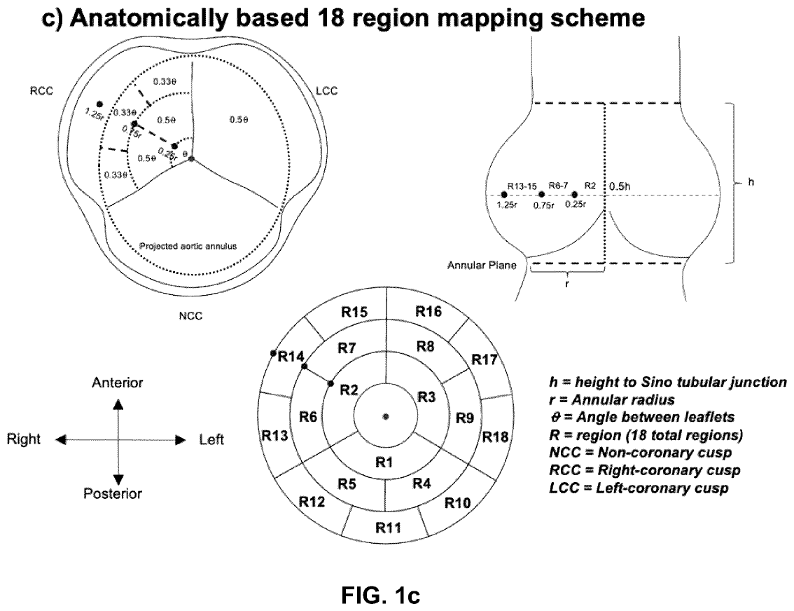

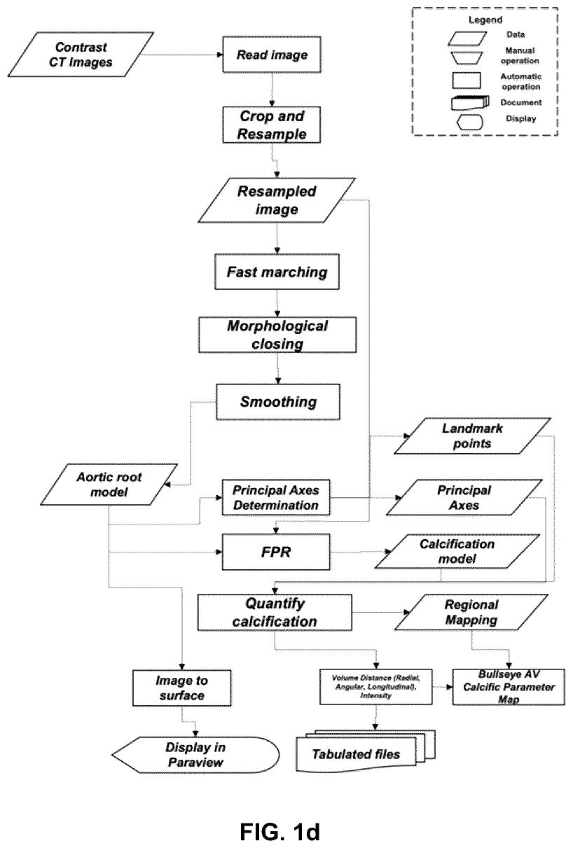

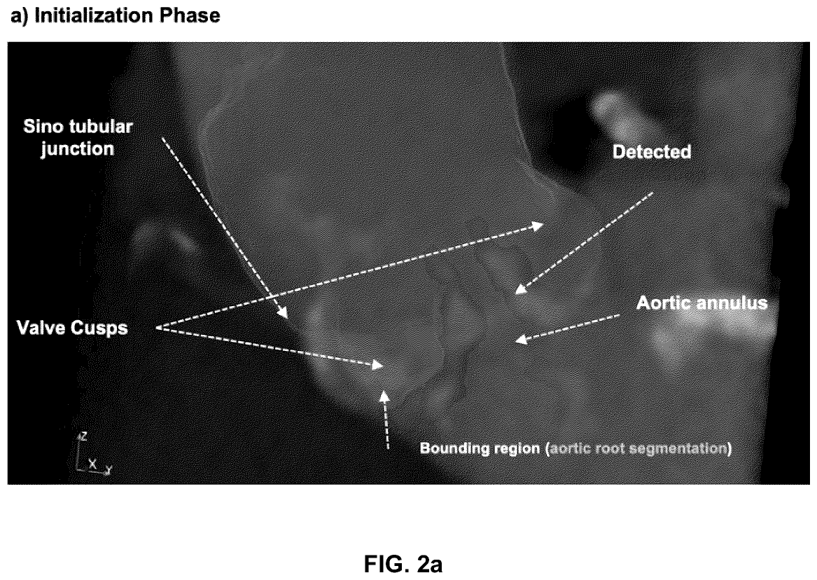

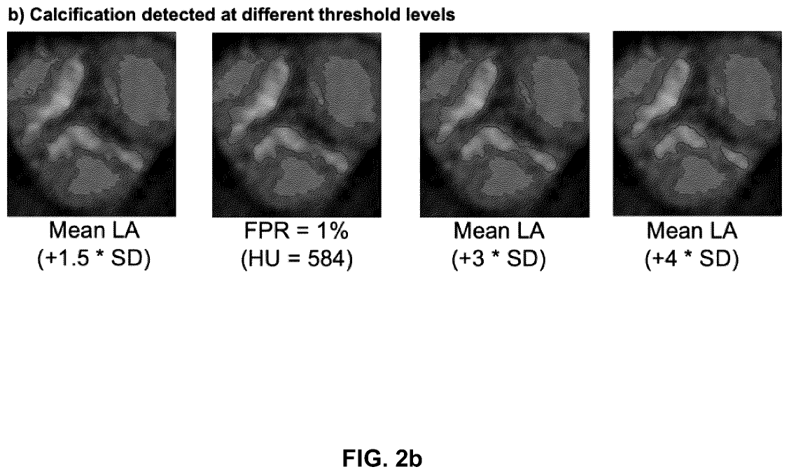

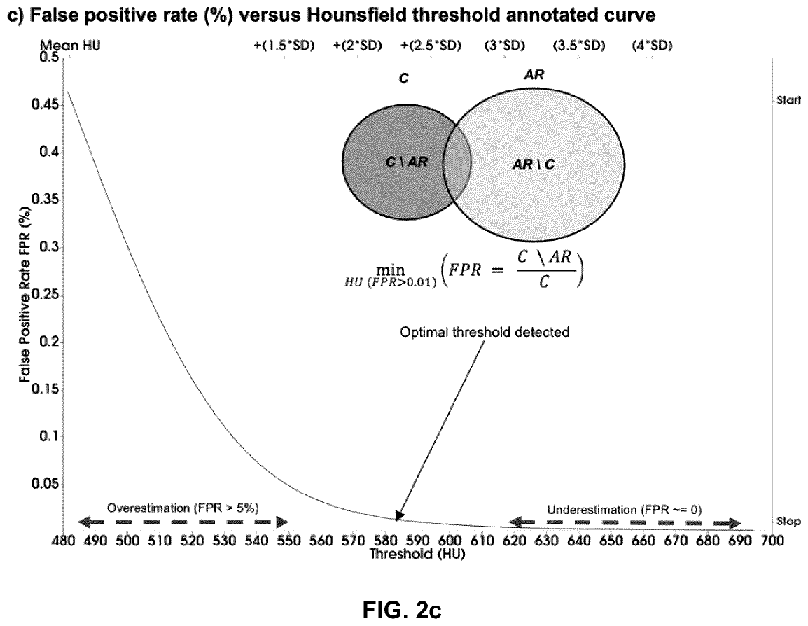

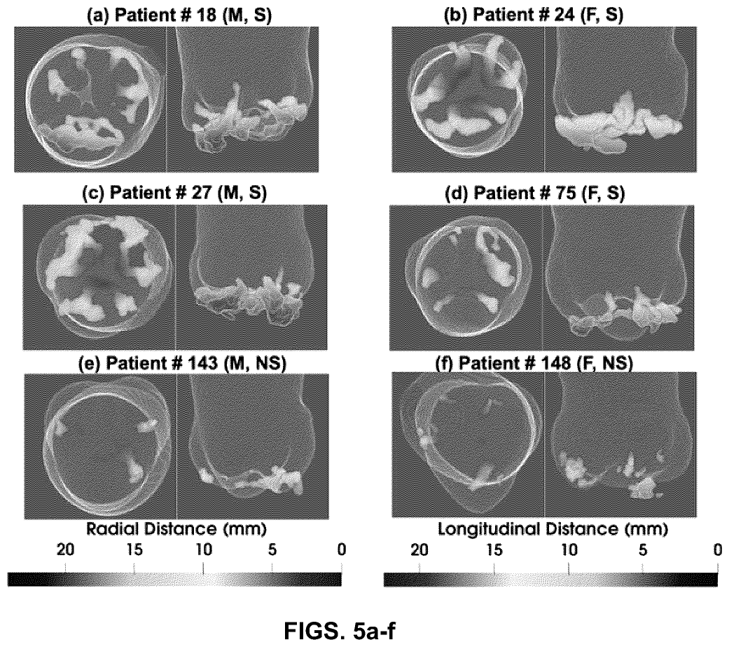

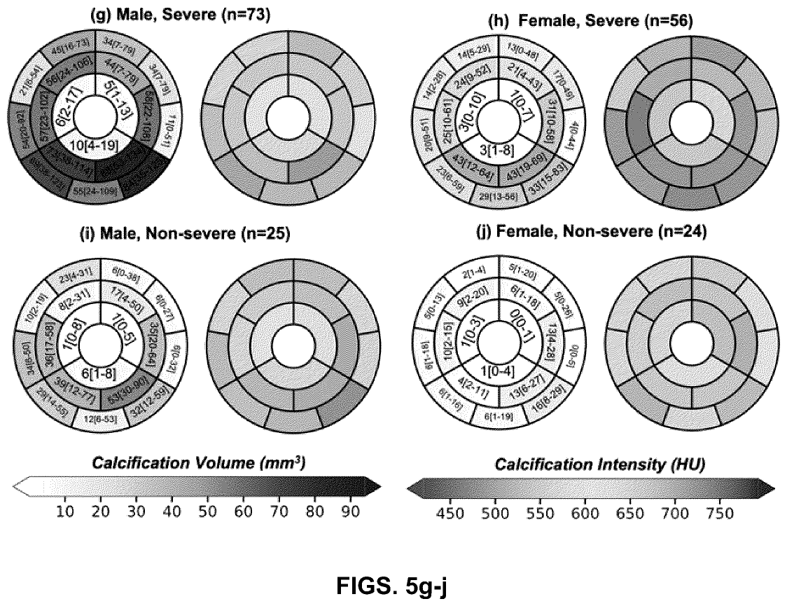

The disclosed systems and methods may include a framework having pipelines designed for the 3D visualization and quantification of calcific lesions using contrast-enhanced thoracic CT images (e.g., FIGS. 1-6 and Tables 1, 4-6). The disclosed calcification detection method may use a novel segmentation scheme that is based on automatically detected local image features (e.g., FIG. 2). The disclosed method may obviate the need for manual annotation of a region of interest in the ascending aorta or a predetermined minimum Hounsfield (HU) cutoff value. The described computational framework may require the field of view to be initially localized to the aortic valve based on the standard assessment guidelines for TAVR planning (e.g., FIG. 1a). A partial aortic root (AR) model may then be automatically segmented (e.g., FIG. 1b). Using the aortic root segmentation, principal directions and landmark points may be generated semi-automatically (e.g., FIG. 1a, 1b). Using this initial set of parameters, a fully automatic detection scheme may be designed to detect the maximal amount of calcific content regardless of the effect of the contrast agent on luminal attenuation (e.g., FIG. 2). This may be accomplished by measuring the false positive rate (FPR) between iterative decrements of HU threshold reconstructions of calcific segments and the partial aortic root model (e.g., FIG. 2). Subsequently, density and topographic maps of both radial and longitudinal measurements for the detected calcific regions may be generated (e.g., FIG. 1b). In addition, detected calcific regions may be further described in terms of global and local geometric shape descriptors. Finally, an anatomical, 18 region mapping scheme (e.g., FIG. 1c) may be developed based on the following standardized measurements: height to sino-tubular-junction, annular area derived radius and angles of the interleaflet triangle (e.g., FIG. 1a). These measurements may be used as distance thresholds (18 regions from coaptation zone to attachment) in order to quantify regional calcific volume and average Hounsfield intensity across the cohort (e.g., FIG. 5, 6b). Further detailed information regarding methodology and theory is described herein below.

The disclosed method was evaluated on a cohort of patients diagnosed with AS in both pre and post intervention. The intervention can be any suitable medical procedure or surgery to address a health condition. For example, and without limitation, the intervention can be TAVR. The scores using the disclosed method on contrast-enhanced images were compared to the standard scores produced for non-contrast [18, 20, 23] images that were scored using commercially available software (Calcium scoring application, Syngo.via; Siemens Healtheneers).

### Study Population

178 patients diagnosed with AS from Hamilton General Hospital (Hamilton, Canada between 2020 and 2022) were retrospectively selected. Non-consecutive data collection included patients evaluated for TAVR with severe or non-severe AS diagnosed by 2D doppler echocardiography and who underwent both gated contrast and non-contrast CT within 3 months of the echocardiogram. Severity was determined by recommended thresholds for aortic valve area, peak aortic jet velocity and mean valve pressure gradient [24]. Waiver of informed consent and data transfer was approved by the Institutional Review Boards of the institution (HiREB), Hamilton General Hospital. The selections were done by operators blinded to the objectives and contents of this disclosure. Clinical measurements were performed per relevant guidelines and regulations including guidelines of the American College of Cardiology and American Heart Association. Demographic and procedural data were collected from the patients' medical records. Table 1 provides the patient characteristics.

TABLE 1
Baseline patients characteristics of study cohort (N = 178). Age
and aortic valve area is presented as median [25th-75th percentile]
for continuous variables and count (%) for categorical variables.

| | Category | Value |
|---|---|---|
| Age (years)-mean+−sd | | 80.5 ± 7.3 |
| Sex-n(%) | Female | 80(44.9%) |
| | Male | 98(55.1%) |
| AS Severity-n(%) | Non severe | 49(27.5%) |
| | Severe | 129(72.5%) |
| NYHA Class-n(%)ₙ₌₂₁₉ | I | 17(9.6%) |
| | II | 70(39.3%) |
| | III | 78(43.8%) |
| | IV | 13(7.3%) |
| Coronary artery disease-n(%)ₙ₌₂₁₃ | Yes | 85(49.4%) |
| Hypertension-n(%)ₙ₌₂₁₅ | Yes | 164(94.8%) |
| Type 2 diabetes mellitus-n(%)ₙ₌₂₀₆ | Yes | 74(78.6%) |
| Dyslipidemia-n(%)ₙ₌₂₁₀ | Yes | 132(78.6%) |
| Atrial fibrillation-n(%)ₙ₌₂₀₄ | Yes | 65(39.9%) |

### CT Acquisition

The patients underwent both contrast and non-contrast enhanced CT scans using a GE Healthcare Discovery CT750 HD Scanner 64 slice 40 mm detector. Contrast CT images were acquired using retrospective gating without tube current modulation of the entire cardiac cycle using a slice thickness of 0.625 mm. Prospectively ECG gated non-contrast CT was performed in a sequential mode at 60%-80% of the RR interval using a slice thickness of 3 mm. Table 2 provides the CT acquisition parameters.

TABLE 2
CT scanning parameters for the study cohort

| | CT scanning parameters |
|---|---|
| Acquisition | Retrospective gated |
| Slice thickness | 0.625 or 3 mm |
| Tube rotation | 0.35 s |
| Pitch | 0.24:1 |
| Tube voltage | 120 kV |
| Tube current | 400-500 mA |
| Reconstruction | Adaptive statistical iterative reconstruction |
| | (ASIR) at 30% |
| Cardiac phase selected | Best diastole (70-75%) cardiac cycle |

The disclosed framework may be based on open-source software, Paraview v.5.9.0 [25] for pre-processing and visualization, complimented by in-house Python modules based on Simple ITK v.2.0.2 [26, 27] for segmentation and quantification of the regions of interest.

Reference is now made to FIGS. 1a-1d and FIGS. 2a-2c. FIGS. 1a-1d show visual representations of an example computational framework for assessing aortic valve leaflet calcification. FIG. 1a—Left: Initial localization of AV; Right: Automatic calcific threshold determination based on automatic estimation of the ratio between non-calcified lumen segments and calcific regions. FIG. 1b—Different quantification color-coded maps from left to right; calcification separated by leaflet 1-3 corresponding to NCC, RCC and LCC respectively; radial distance map with range of [0-annular radius (mm)]; longitudinal distance map with range of [0-height to sino-tubularjunction (mm)]; HU intensity map for calcific lesions detected, color mapped by a fixed Hounsfield intensity range of 484-1585 HU. The color bar is partitioned into 4 sections with 275.25 HU difference for each partition. FIG. 1c—Schematic diagram of disclosed anatomically based 18 region model of the aortic valve, regions are counted in clockwise order starting from the non-coronary cusp. Region subdivisions are based on parametric coordinates in cylindrical coordinate system, normalized by patient specific aortic root dimensional measurements (STJ height h, annular area derived radius r and angles θ between the interleaflet edges measured from the “en-face” short axis view of the valve. FIG. 1d—flowchart showing the example computational framework for assessing aortic valve calcification. The flowchart describes a series of operations, either manual or automatic that operate on an initial input set of CT images, to eventually generate tabulated files with relevant clinical indices such as volume scores and display 3D models of detected valve and calcification.

FIGS. 2a-2c show visual representations of an example calcification detection algorithm that may be used with the disclosed framework. FIG. 2a-Initialization step with automatically detected aortic root segmentation and “en-face” short axis view of leaflets. FIG. 2b—2D short-axis section of the aortic valve with overlaid contours of detected calcium segments in red and partial aortic root lumen in cyan using different described methods. FIG. 2c—FPR vs HU threshold curve measuring the rate of calcific pixels classified as non-calcific lumen segments, an optimal threshold in a wide HU band is determined such that the resulting calcific segmentation is not under estimated or over estimated.

### Image Pre-Processing

Initially, the volumetric CT images, with acquisition parameters detailed in Table 2 may be loaded into Paraview and the opacity transfer function in the volumetric view may be adjusted to highlight soft tissue in the thoracic CT scan, such as vessels and organs. With the aorta clearly visible, the image may then be cropped to have a field of view focused on the aortic root, including the interface between the aortic valve and the left ventricular outflow tract (LVOT), a part of the ascending aorta after the Sino-tubular junction (STJ) and excluding any other surrounding structures. The field of view may then be resampled to have isotropic voxel dimensions of [0.5, 0.5, 0.5 mm].

### Valve Segmentation

To segment the valve from surrounding tissue, the fast-marching method may be employed. The fast-marching method is closely related to level-set segmentation which is based on solving a partial differential equation (PDE) named the Eikonal equation (Equation 1) [28]

|Δu|=C (Equation 1)

where u describes the evolution of a closed surface as a function of time and C represents the cost image which is modeled as the speed of the propagating surface. The fast-marching method has been shown to segment anatomical regions of complex topology and varied curvature [28]. The segmentation process is controlled by seed points where it propagates outward from the local surface normal of the seed points]28]. The propagation is controlled by the design of what is known as the speed image or function which aims to incorporate image features such that high values are present near boundaries and lower values in homogenous regions [28]. For the disclosed framework, a smoothed gradient magnitude image may be first derived using a gradient magnitude recursive gaussian operation [29] with a smoothing sigma of 0.5. The final speed image may then be calculated using a bounded reciprocal operation [29] which produces a new image with values of zero near the boundaries or edges, and values of one in homogeneous regions. The determination of the cost function can be defined as in Equation 2:

$$C ( x ) = \frac{1}{1 + \vert ∇ {I}_{g} ( x ) \vert} \qquad ( Equation 2 )$$

where C(x) denotes the cost and I_{g}(x) represents the image gradient at some index x. The centroid of the previously annotated field of view may be used as initial seed point, along with the constructed speed image as inputs into a fast-marching procedure [29]. The output of the fast-marching procedure may be a time-crossing map, which indicates the time of arrival of the propagated level-set front. This arrival time can effectively become the number of iterations required to segment a particular structure. A threshold for this output may be set to a fixed time/level using a binary threshold of 400 iterations, which, may be an appropriate value for segmenting the aortic root shape without the calcification in the leaflets. An important implication of how the cost function was designed may be that the segmentation propagates through luminal non-calcified segments that are smoother in the speed image and stop at calcified “sharp” edges in the gradient function.

Finally, to correct for noise and holes in the segmentation and obtain a smoothed surface, a morphological closing operation [29] with a kernel radius of 2 voxels may be initially used, and then a smoothing recursive gaussian operation [29] with a kernel radius of 2 voxels may be used. For visualizing the binary volume as a transparent surface, the contour filter [30] in Paraview on the final filter output may be used. The detailed parameterization for the various filters is presented in Table 3.

TABLE 3
Overview of operations/filters used in the platform, input, output and parameters used where
applicable

| | Procedure | Input | Value | Output |
|---|---|---|---|---|
| 1 | Crop and Resample | Size | Patient specific | Resampled Image |
| | | Resolution | 0.5 mm | (Image [HU]) |
| 2 | Fast marching | Speed Sigma | 0.65 | Binary mask |
| | | No. of iterations | 300 | |
| | | Seed point location | Center of aortic root (1) | |
| 3 | Morphological | Kernel radius | 2 | Binary mask |
| | closing | | | |
| 4 | Smoothing | Sigma | 0.5 | Aortic root model |
| | | Kernel radius | 2 | (Binary mask) |
| 5 | Principal Axes | Aortic root model | Binary mask (4) | Direction matrix |
| | Determination | | | Shape boundary |
| | | | | Geometric center |
| 6 | FPR | Aortic root model | Binary mask (4) | Calcification model |
| | | Resampled image | Image [HU] (1) | (Binary mask) |
| | | False Positive Rate | 0.01 = 1% | |
| | | Threshold | | |
| 7 | Quantify calcification | Calcification model | Binary mask (6) | Regional calcification map |
| | | | | (Binary mask) |
| | | Principal axes | 3 × 3 direction matrix (5) | Radial distance map |
| | | orientation | | (Image [mm]) |
| | | Landmark points | 4 points (5) | Longitudinal distance map |
| | | | | (Image [mm]) |
| | | Volume | Binary mask (6) | Calcification intensity map |
| | | | | (Image [HU]) |
| 8 | Regional Mapping | STJ Height | Patient specific | Anatomical 18 Region |
| | | Annular Radius | Derived from patient | Volume Map |
| | | | specific annular area | |
| | | Interleaflet Triangle | Patient specific | Anatomical 18 Region |
| | | Angles | | Average Intensity Map |
| | | Calcification Model | Binary mask (6) | |
| | | Principal axes | 3 × 3 direction matrix (5) | |
| | | orientation | | |

### Principal Axes Determination and Landmark Points

After the smoothed aortic root model is constructed, the shape features of the binary segmentation may be analyzed. The analysis may be performed through shape statistics algorithm [31] which can enable automatic estimation of the principal orientation axes, the center of mass, and the boundary points of a binary object based on its topology. These principal axes may correspond to anatomical short axis view, and two long axis views that are perpendicular to the “En-face” short axis view. These three orientation vectors can be manually corrected by a user, if necessary, to properly orient the desired anatomical views.

The completion of the analysis may require annotation of four landmark points. The first landmark point may be initialized from the center of mass of the aortic root model and may then be corrected such that it falls at the level of the STJ plane and centered at the aortic annulus in the short-axis orientation. Next, using the estimated position of the center of the aortic root, the boundary points and the short-axis direction, the maximum elevation points directed in a direction towards the LVOT may be located. These points may then be filtered to localize probable location of valve hinge points. The initial location of these three points may be corrected manually, such that the points are centered at each cusp and ordered sequentially as (NCC, RCC, LCC) respectively.

The final STJ point, and the orientation axes may together uniquely define a new local coordinate system that is relative to the aortic valve leaflets. The basis vectors of this coordinate system can be computed as an affine transformation (Equation 3), consisting of a rotational component (Equation 4) and a translational component (Equation 5).

$$A = ( \begin{matrix}R \\ 0 … 0\end{matrix} \vert \begin{matrix}b \\ 1\end{matrix} ) \qquad ( Equation 3 )$$

such that

$$R = {R}_{Z} ( γ ) {R}_{Y} ( β ) {R}_{X} ( α ) = \qquad ( Equation 4 )$$

$$[ \begin{matrix}cos γ & - sin γ & 0 \\ sin γ & cos γ & 0 \\ 0 & 0 & 1\end{matrix} ] [ \begin{matrix}cos β & 0 & sin β \\ 0 & 1 & 0 \\ - sin β & 0 & cos β\end{matrix} ] [ \begin{matrix}1 & 0 & 0 \\ 0 & cos α & - s in α \\ 0 & sin α & cos α\end{matrix} ]$$

$$and$$

$$b = [ \begin{matrix}{C}_{x} \\ {C}_{y} \\ {C}_{z}\end{matrix} ] \qquad ( Equation 5 )$$

The original frame of reference may be represented as x, y, z corresponding to anatomic sagittal, coronal, and axial views, respectively. The rotation angles required to transform the original frame of reference to the local frame may be determined from the final “En-face” short axis plane normal, yielding rotations about the original frame with angles α, β, γ respectively about each axis (Equation 4). The translation vector b may be determined from STJ landmark point defined as cₓ, c_{y}, c_{z}. The entire affine transformation may be later used to realign the image direction, so that the long axis of the valve is now parallel to the unit z and leaflet faces are parallel to the z=0 plane. This may be necessary to accurately determine the spatial position of calcific voxels for distance map generation.

### Calcium Detection (FPR Method)

When viewing the AV region of interest (ROI) in cases of AS from CT images, disconnected groups of calcific lesions can be observed. These lesions may be presented as relatively brighter intensities compared to tissue and lumen. The presence of the contrast agent can make the lower and upper bounds of these intensities practically unknown which can be in part due to differences in contrast absorption for each patient [32]. Therefore, given that the calcific intensity average will be statistically higher than surrounding objects, a procedure that can grow iteratively from an initial HU estimate may be required. The initial estimate of calcific HU threshold is based on average HU of the partial aortic root model. The disclosed method may proceed based on the following steps. Using the initial HU estimation, the HU threshold may be iteratively increased in steps of 1 HU unit. This may be performed exhaustively until a minimum false positive rate criteria is reached (1%).

$$F P R = \frac{C ∖ A R}{C} \qquad ( Equation 6 )$$

The (FPR) may be defined [33] in set notation as: ratio between, the no. of pixels in the set difference (\) of the aortic root model (AR) segmentation from the calcification model (C) and the total no. of pixels in the calcification model. As the threshold is increased, this rate may decrease as the number of luminal pixels mislabeled as calcific decreases. This representation may yield a non-linear relationship between the rate and HU threshold of detection which can then be treated as a constrained global minimization problem. However, the discrete function representation may not have a global minimum and a specific constraint may be required to be set to avoid overshooting an optimal threshold. In some embodiments, a minimum of 0.01/1% FPR may be used to stop the iterations and find the optimal threshold. The iterative scheme combined with a global constraint can yield calcific detection thresholds that are robust to variations in intraluminal contrast, calcific density and other image specific factors. The parameters used to initialize the shape overlap algorithm are presented in Table 3.

### Volume and Geometric Analysis

The final segmented calcium region may be presented as a binary image with values of one in pixels where calcium was detected and values of zero in pixels lacking calcium. The volume score may be determined based on Equation 7:

$${V}_{S} = ( {\sum }_{i = 0}^{N} {M}_{i} ) * 0 . {5}^{3} \qquad ( Equation 7 )$$

The volume score Vs may be calculated by multiplying the total count of ones present on the binary image matrix with the image spacing where Mᵢ represents the value of the pixel at index i and N is the total number of pixels. The image spacing can be 0.5 mm in each direction due to the previous uniform resampling, giving a total volume score in mm³.

Since the image volume may now be oriented with the long axis of the valve parallel to the z unit vector, topographic distance maps of the segmented calcium volume can be generated by first converting all pixel coordinates into a physical cartesian coordinate space using the conversion in Equation 8:

$$( \begin{matrix}{P}_{1} \\ {P}_{2} \\ {P}_{3}\end{matrix} ) = ( \begin{matrix}{O}_{1} \\ {O}_{2} \\ {O}_{3}\end{matrix} ) + ( \begin{matrix}1 & 0 & 0 \\ 0 & 1 & 0 \\ 0 & 0 & 1\end{matrix} ) * ( \begin{matrix}0.5 & 0 & 0 \\ 0 & 0.5 & 0 \\ 0 & 0 & 0.5\end{matrix} ) * ( \begin{matrix}{I}_{1} \\ {I}_{2} \\ {I}_{3}\end{matrix} ) \qquad ( Equation 8 )$$

where \vec{P} represents the resulting physical space position of an image pixel at some index \vec{I}, and \vec{O} is the physical space origin of the first image index. This transformation may be automatically handled using the function transform index to physical point [29] and repeated for each pixel index in the image volume. Next, the basis of each physical coordinate may be changed to the previously defined local aortic valve system. This may be accomplished by a simple dot product with the transformation matrix that defines the new system (Equation 9):

$$( \begin{matrix}{{P}_{1}}^{′} \\ {{P}_{2}}^{′} \\ {{P}_{3}}^{′} \\ 1\end{matrix} ) = A . ( \begin{matrix}{P}_{1} \\ {P}_{2} \\ {P}_{3} \\ 1\end{matrix} ) \qquad ( Equation 9 )$$

A conversion from cartesian to cylindrical coordinates can then be applied to \vec{P} for each image pixel, using the transformation in Equation 10:

$$r = \sqrt{{{P}_{1}}^{′2} + {{P}_{2}}^{′2}} ; θ = {tan}^{- 1} ( \frac{{{P}_{2}}^{′}}{{{P}_{1}}^{′}} ) ; l = {P}_{3}^{′} \qquad ( Equation 10 )$$

These transformations may enable the generation of both radial and longitudinal distance maps relative to the aortic sinuses landmark point. Image pixel values may be set as r, θ or l for radial, angular, and longitudinal maps respectively. These new distance image maps can be then used to introduce the anatomical regional mapping scheme to precisely quantify calcific content via the schematic described in FIG. 1c. The regional map may rely on successive binary thresholding operations followed by a Boolean intersection (&) operation where in this case the thresholds may be determined from the patient specific dimensions of the STJ Height (h), Annular area derived radius (r) and the specific interleaflet triangle angles that demarcate the angular extent of each leaflet (θ_{ncc}, θ_{rcc}, θ_{lcc}). The specific thresholds used as per the proposed 18 region map scheme (FIG. 1c) may be explicitly described in Equation 11. The specific percentage multipliers and patient specific ranges may be based on common presentation of a normal tricuspid valve anatomy [34] and may aim to equalize the area of each region to facilitate comparison with increasing angle divisions while moving towards the attachment from the coaptation zone and fixed radial intervals 0.5r apart.

$${R}_{n} = \{ \begin{matrix}{R}_{1} ( Σ {R}_{i} \& {ΣΛ}_{i} \& Σ {L}_{i} ) s . t : 0 \leq {R}_{i} < 0.25 r ; 0 \leq {Λ}_{i} < {θ}_{ncc} ; 0.5 h \leq {L}_{i} < h \\ {R}_{2} ( Σ {R}_{i} \& {ΣA}_{i} \& Σ {L}_{i} ) s . t : 0 \leq {R}_{i} < 0.25 r ; {θ}_{ncc} \leq {A}_{i} < {θ}_{rcc} + {θ}_{ncc} ; 0.5 h \leq {L}_{i} < h \\ {R}_{3} ( Σ {R}_{i} \& {ΣA}_{i} \& Σ {L}_{i} ) s . t : 0 \leq {R}_{i} < 0.25 r ; {θ}_{rcc} + {θ}_{ncc} \leq {A}_{i} < {θ}_{rcc} + {θ}_{ncc} + {θ}_{lcc} ; 0.5 h \leq {L}_{i} < h \\ {R}_{4} ( Σ {R}_{i} \& {ΣA}_{i} \& Σ {L}_{i} ) s . t : 0.25 r \leq {R}_{i} < 0.75 r ; 0 \leq {A}_{i} < 0.5 {θ}_{ncc} ; 0.5 h \leq {L}_{i} < h \\ {R}_{5} ( Σ {R}_{i} \& {ΣA}_{i} \& Σ {L}_{i} ) s . t : 0.25 r \leq {R}_{i} < 0.75 r ; 0.5 {θ}_{ncc} \leq {A}_{i} < {θ}_{ncc} ; 0.5 h \leq {L}_{i} < h \\ {R}_{6} ( Σ {R}_{i} \& {ΣA}_{i} \& Σ {L}_{i} ) s . t : 0.25 r \leq {R}_{i} < 0.75 r ; {θ}_{ncc} \leq {A}_{i} < 0.5 {θ}_{rcc} + {θ}_{ncc} ; 0.5 h \leq {L}_{i} < h \\ {R}_{7} ( Σ {R}_{i} \& {ΣA}_{i} \& Σ {L}_{i} ) s . t : 0.25 r \leq {R}_{i} < 0.75 r ; 0.5 {θ}_{rcc} + {θ}_{ncc} \leq {A}_{i} < {θ}_{rcc} + {θ}_{ncc} ; 0.5 h \leq {L}_{i} < h \\ {R}_{8} ( Σ {R}_{i} \& {ΣA}_{i} \& Σ {L}_{i} ) s . t : 0.25 r \leq {R}_{i} < 0.75 r ; {θ}_{rcc} + {θ}_{ncc} \leq {A}_{i} < {θ}_{rcc} + {θ}_{ncc} + 0.5 {θ}_{lcc} ; 0.5 h \leq {L}_{i} < h \\ {R}_{9} ( Σ {R}_{i} \& {ΣA}_{i} \& Σ {L}_{i} ) s . t : 0.25 r \leq {R}_{i} < 0.75 r ; {θ}_{rcc} + {θ}_{ncc} + 0.5 {θ}_{lcc} \leq {A}_{i} < {θ}_{rcc} + {θ}_{ncc} + {θ}_{lcc} ; 0.5 h \leq {L}_{i} < h \\ {R}_{10} ( Σ {R}_{i} \& {ΣA}_{i} \& Σ {L}_{i} ) s . t : 0.75 r \leq {R}_{i} < 1.25 r ; 0 \leq {A}_{i} < 0.33 {θ}_{ncc} ; 0.5 h \leq {L}_{i} < h \\ {R}_{11} ( Σ {R}_{i} \& {ΣA}_{i} \& Σ {L}_{i} ) s . t : 0.75 r \leq {R}_{i} < 1.25 r ; 0.33 {θ}_{ncc} \leq {A}_{i} < 0.66 {θ}_{ncc} ; 0.5 h \leq {L}_{i} < h \\ {R}_{12} ( Σ {R}_{i} \& {ΣΛ}_{i} \& Σ {L}_{i} ) s . t : 0.75 r \leq {R}_{i} < 1.25 r ; 0.66 {θ}_{ncc} \leq {Λ}_{i} < {θ}_{ncc} ; 0.5 h \leq {L}_{i} < h \\ {R}_{13} ( Σ {R}_{i} \& {ΣΛ}_{i} \& Σ {L}_{i} ) s . t : 0.75 r \leq {R}_{i} < 1.25 r ; {θ}_{ncc} \leq {Λ}_{i} < 0.33 {θ}_{rcc} + {θ}_{ncc} ; 0.5 h \leq {L}_{i} < h \\ {R}_{14} ( Σ {R}_{i} \& {ΣA}_{i} \& Σ {L}_{i} ) s . t : 0.75 r \leq {R}_{i} < 1.25 r ; 0.33 {θ}_{rcc} + {θ}_{ncc} \leq {A}_{i} < 0.66 {θ}_{ncc} + {θ}_{ncc} ; 0.5 h \leq {L}_{i} < h \\ {R}_{15} ( Σ {R}_{i} \& {ΣA}_{i} \& Σ {L}_{i} ) s . t : 0.75 r \leq {R}_{i} < 1.25 r ; 0.66 {θ}_{rcc} + {θ}_{ncc} \leq {A}_{i} < {θ}_{rcc} + {θ}_{ncc} ; 0.5 h \leq {L}_{i} < h \\ {R}_{16} ( Σ {R}_{i} \& {ΣA}_{i} \& Σ {L}_{i} ) s . t : 0.75 r \leq {R}_{i} < 1.25 r ; {θ}_{rcc} + {θ}_{ncc} \leq {A}_{i} < {θ}_{rcc} + {θ}_{ncc} + 0.33 {θ}_{lcc} ; 0.5 h \leq {L}_{i} < h \\ {R}_{17} ( Σ {R}_{i} \& {ΣA}_{i} \& Σ {L}_{i} ) s . t : 0.75 r \leq {R}_{i} < 1.25 r ; {θ}_{rcc} + {θ}_{ncc} + 0.33 {θ}_{lcc} \leq {Λ}_{i} < {θ}_{rcc} + {θ}_{ncc} + 0.66 {θ}_{lcc} ; 0.5 h \leq {L}_{i} < h \\ {R}_{18} ( Σ {R}_{i} \& {ΣA}_{i} \& Σ {L}_{i} ) s . t : 0.75 r \leq {R}_{i} < 1.25 r ; {θ}_{rcc} + {θ}_{ncc} + 0.66 {θ}_{lcc} \leq {A}_{i} < {θ}_{rcc} + {θ}_{ncc} + {θ}_{lcc} ; 0.5 h \leq {L}_{i} < h\end{matrix}$$

where R, A and L are distance map images with values for each pixel i defined as the radial, angular and longitudinal distance respectively (Equation 10) relative to a point located at the STJ plane and centered in relation to the aortic annulus. The procedure can find all pixels that satisfy the cylindrical thresholding criteria in each dimension and then can combine all the identified pixels via an intersection Boolean operation &. Examples of 3D distance image views of the quantification maps on top of the valve surface and the region of interest, are shown in FIGS. 5(a)-(f). Examples of the final conformal representation of the measured regional quantities is presented in FIGS. 5(g)-(j). For each calcific lesion detected the shape features may be analyzed through a shape statistics algorithm [31] which can enable automatic estimation of the principal orientation axes, the center of mass, the boundary points, the roundness, the flatness, the elongation, the equivalent spherical radius, the equivalent ellipsoidal diameters and the fractal dimension index of a calcific lesion based on its topology.

### Normalization of the Metrics

For normalizing the volume scores (V_{sd}) of the contrast scores, an approximation of annular radius from the annular area that was measured using CT for all patients may be defined as follows [35]:

$$Indexed Contrast Calcific Volume = \frac{{V}_{S}}{Annular area} \qquad ( Equation 12 )$$

The annular radius of a circular aortic annulus using the area may then be estimated as follows:

$$Annular radius = \frac{\sqrt{\frac{Annular area}{π}}}{2} \qquad ( Equation 13 )$$

The normalization procedures may be critical to interpret the described metrics in the presence of variabilities in valve dimensions, valve morphology, patient size and gender variability [18, 35].

### Validation of the Disclosed Systems and Methods

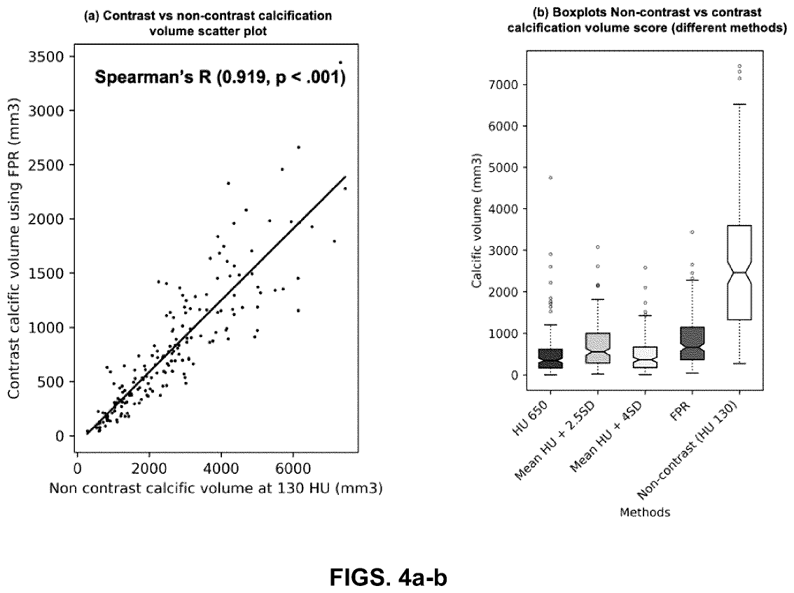

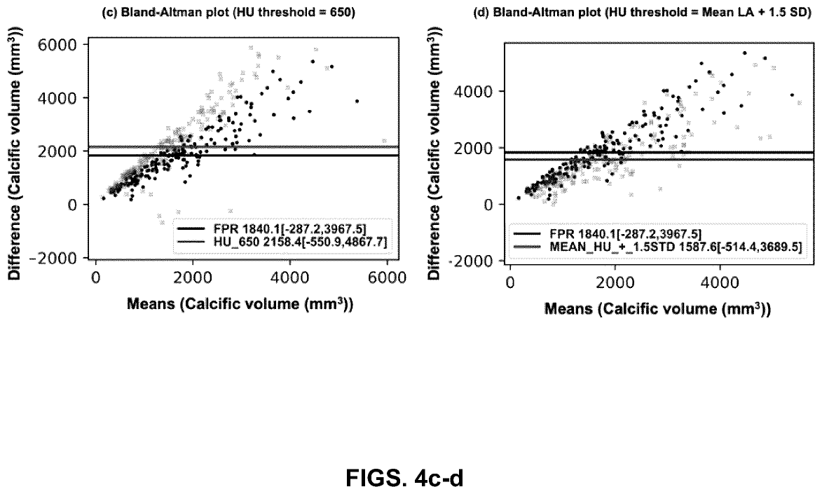

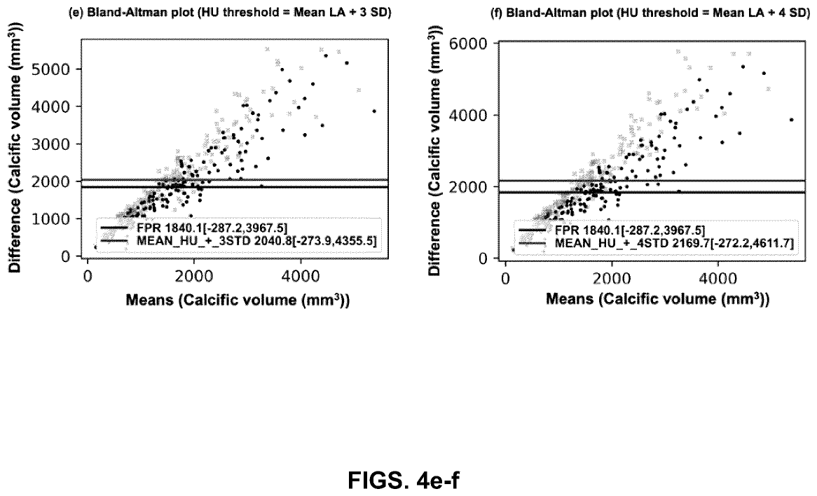

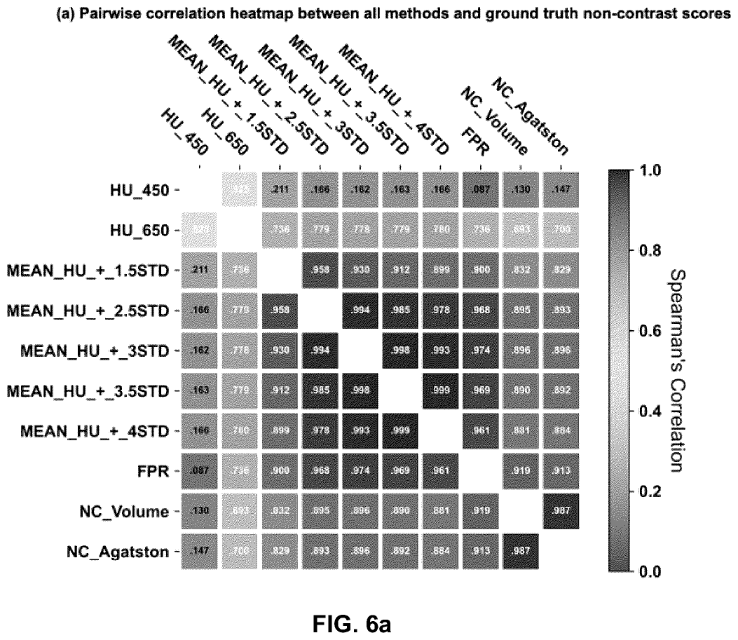

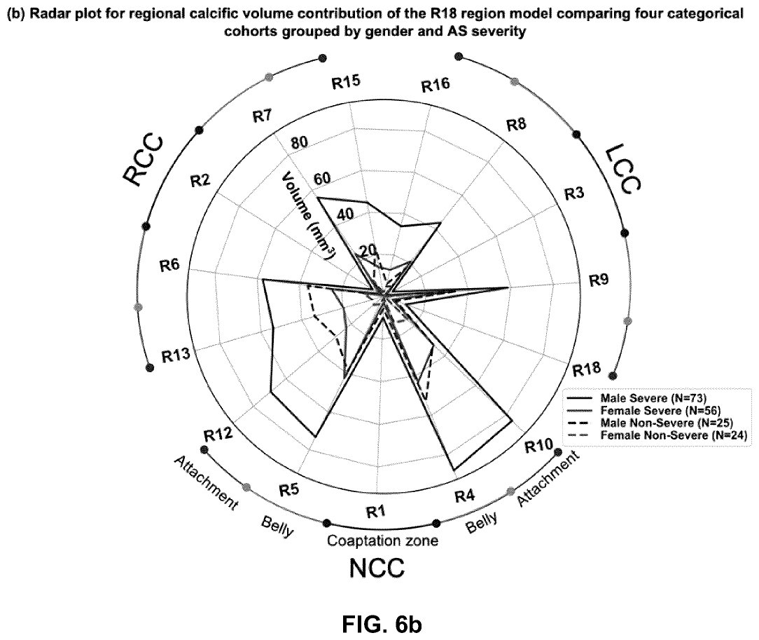

Appropriate statistical tests were performed using Jamovi v.1.8. Summaries of the variables and tests performed in (FIG. 4, FIG. 6; Table 4; Table 5; Table 6) are outlined as follows. Correlations and comparisons between the variables was performed using Spearman's rank correlation test, Wilcoxon rank paired t-test, non-parametric One-way ANOVA (Kruskal-Wallis) followed by post-hoc Dwass-Steel-Critchlow-Fligner pairwise comparisons where applicable. Bland-Altman analysis of difference was used to compare the disclosed FPR method alongside the conventional methods based on a fixed HU threshold (650) or a mean HU attenuation at the ascending aorta (+1.5, +2.5, +3, +3.5,+4 SD) (FIG. 4; FIG. 6a). Values were reported as median [25^{th} —75^{th} percentile], mean±standard deviation or mean difference [lower limit of agreement, upper limit of agreement] where applicable. Finally, interobserver variability was measured using the intraclass correlation coefficient (ICC; two-way random agreement) for a sub cohort of 49 patients. Statistical significance was considered when the p-value was less than 0.05.

### Example Results

The study consisted of patients diagnosed with non-severe (49/178; 27.5%) and severe (129/178; 72.5%) AS. The cohort had an average age of 80.5±7.3 years and included males (98/178; 55.1%) and females (80/178; 45.9%). Baseline characteristics are presented in Table 1. Interobserver variability was assessed on a random sub-group of 49 patients using intraclass correlation coefficient (ICC; two-way random agreement) showing excellent reproducibility (ICC:0.95; 95% Cl 0.94-0.96; raters=2).

### FPR Method Performance in Contrast Images

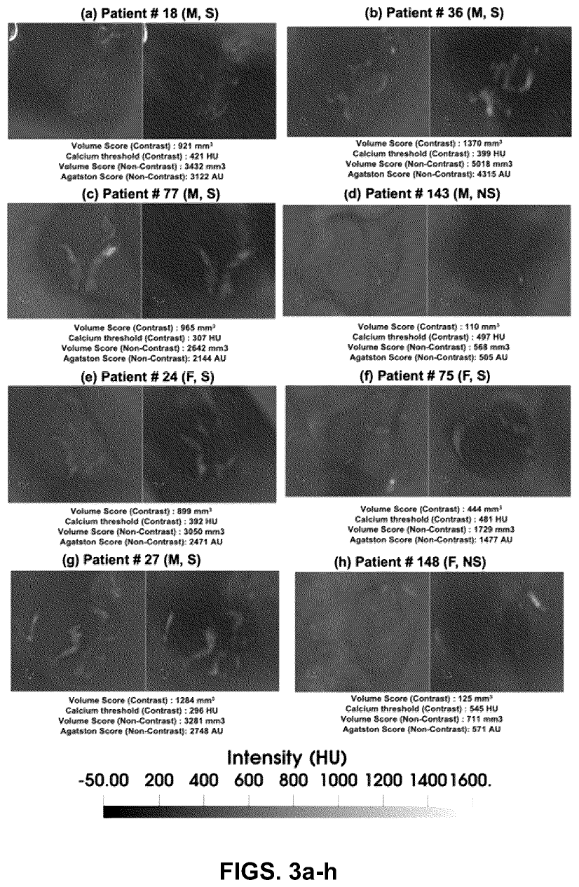

Reference is now made to FIGS. 3a-3h showing example results of detected calcification in contrast versus non-contrast representative cases, using the disclosed framework. Eight patients were selected around the median volume score grouped by sex (M-F) and AS severity (NS-S). The left panel shows the detected calcification in the same patient using contrast enhanced images. The right panel shows the detected calcification in the same patient using non-contrast images. Field of view, orientation and opacity mapping matching was performed to facilitate comparison.

The contrast and non-contrast segmented calcium volumes were compared for the 8 representative patient samples in FIG. 3. By focusing the field of view on the aortic valve in both contrast and non-contrast images, the patterns of calcification detected between the contrast (FPR method) and non-contrast (standard 130 HU) can be qualitatively compared. The detected calcification is identified with a red outline around the region of interest in each subplot (FIG. 3).

Reference is now made to FIGS. 4a-4f showing example statistics and data plots for parameters obtained using the disclosed framework. FIG. 4a—Scatter plot of contrast versus non-contrast calcific volume. FIG. 4b—Boxplot comparing contrast (different methods) versus non-contrast calcific volume FIGS. 4c-4f—Bland-Altman plot of differences.

In general, a strong agreement of detected calcification between both images in terms of the shape and location can be observed. Additionally, calcification volume using the FPR method showed the best correlation against non-contrast volume and Agatston scores (r=0.919; p<0.001) (r=0.913; p<0.001) in contrast-enhanced images (FIG. 4a; FIG. 6a). With regards to absolute volume of detected calcification, a large difference is observed. Contrast images consistently produce a lower total volume of detected calcification compared to the non-contrast images. Paired comparisons (FIG. 3; FIG. 4b; Table 4) showed significantly lower volume scores for the contrast images compared to non-contrast volume scores. This underestimation was observed for all methods evaluated against non-contrast (FIG. 4b). Visual differences between detected calcific volumes using the disclosed method and non-contrast images (130 HU threshold) are presented in FIG. 3. Given the systemic bias with non-contrast, methods based on fixed HU or dynamic HU thresholds using luminal attenuation were compared with Bland-Altman agreement plots (FIG. 4c-f), all against the same reference non-contrast volume scores. All evaluated methods showed a proportional bias that increases at higher calcium volumes when compared to non-contrast (FIG. 4c-d) with mean LA+3SD being the closest method to the FPR (r=0.974; p<0.001) (FIG. 6a). Moreover, comparative analysis showed the FPR method had the best overall performance in terms of both low and high calcification volumes with a mean bias (1840.1) and an error at the low and high levels of agreement [−1 0.96SD: −287.2-+1 0.96SD: 3967.5]. Compared to the fixed SD methods, it can be clearly observed that at higher SD, bias increases at high level of agreement as the threshold becomes over-conservative in capturing calcific segments. Alternatively, at lower SD, bias increases at the low level of agreement as the determined threshold overestimates calcific segments.

TABLE 4
Summarized statistics (N = 178). Grouped by sex and AS severity. Presented as
median [25th-75th percentile] for each subgroup.

| | Female | | Male | |
|---|---|---|---|---|
| Parameter | Non_{(n = 24)} | Severe_{(n = 56)} | Non_{(n = 25)} | Severe_{(n = 73)} |
| Contrast CT calcific | 148[115-209] | 473[374-759] | 538[302-737] | 1133[780-1483] |
| volume | | | | |
| (mm³) | | | | |
| Indexed contrast CT | 39[26-57] | 125[84-215] | 114[61-143] | 215[172-302] |
| calcific volume | | | | |
| (mm³/cm²) | | | | |
| FPR Derived Calcific | 462[423-533] | 443[391-591] | 424[348-491] | 375[307-415] |
| Threshold | | | | |
| (HU) | | | | |
| Non-Contrast Calcific | 834[615-1094] | 20142[1434-2878] | 1716[1374-2227] | 3281[2672-4363] |
| Volume | | | | |
| (mm³) | | | | |
| Non-Contrast Agatston | 707[516-867] | 1570[1197-2205] | 1445[1145-1820] | 2645[2094-3460] |
| Score | | | | |
| (AU) | | | | |
| Contrast CT calcific | 64[26-102] | 209[136-353] | 246[123-312] | 449[304-571] |
| volume-NCC | | | | |
| (mm³) | | | | |
| Contrast CT calcific | 36[24-65] | 122[80-253] | 114[85-151] | 343[210-483] |
| volume-RCC | | | | |
| (mm³) | | | | |
| Contrast CT calcific | 44[29-96] | 153[76-222] | 167[113-239] | 300[209-494] |
| volume-LCC | | | | |
| (mm³) | | | | |
| Annular area | 4.0[3.3-4.5] | 4.1[3.5-4.5] | 5.2[4.6-5.6] | 5.1[4.5-5.6] |
| (cm²) | | | | |
| Annular radius | 11.3[10.2-12] | 11.4[10.6-12] | 12.9[12.1-13.3] | 12.7[12.0-13.4] |
| (mm) | | | | |
| STJ Height | 19.7[18-21] | 20.5[19.1-22.5] | 23.6[21-25.6] | 23.4[21.1-25.5] |
| (mm) | | | | |

Finally, the behavior of the different methods can also be observed on a representative severe AS patient in FIG. 2b; FIG. 2c with high thresholds (+3*SD, +4*SD) missing small calcific nodules and low thresholds (+1 0.5*SD) merging large calcific regions between the leaflets. It may be highlighted that luminal attenuation derived methods can over or underestimate calcific deposition, especially in situations of high variance in the blood pool HU intensity distribution or low calcific densities in non-severe cases [18, 21]. The disclosed FPR method can demonstrably compensate for these limitations, by iterative refinement of HU threshold based on an image specific false positive rate. This has implications for both a diagnostic use case of contrast-enhanced calcification score and accurate regional assessment of calcific nodules. Precise regional assessment cannot be performed in non-contrast CT due to low contrast and spatial resolution. This is critical as it may provide an incremental improvement over current qualitative criteria of calcification assessment in TAVR planning [12].

### Pattern and Quantity of Leaflet Calcification in Contrast Images

Reference is now made to FIGS. 5a-5h showing example patterns of calcification in AS for contrast representative cases using the disclosed framework. FIGS. 5a-5f—Six patients were selected around the median volume score grouped by sex (M-F) and AS severity (NS-S). The left panel shows the detected calcification, color mapped by a normalized radial distance range of [0-25.4 mm]. The right panel shows the detected calcification, color mapped by a normalized longitudinal distance range of [0-23.4 mm]. Regional volume, annular center, and annular plane proximity (%) were calculated for each leaflet, termed as non-coronary cusp (N), right coronary cusp (R) and left-coronary cusp (L) respectively. FIGS. 5g-5j—Bullseye aortic valve plot with 18 region map described in FIG. 1c, median values for each categorical group (sex and severity) were used to color the volume and HU intensity map. For the volume maps median [IQR] values were annotated inside the borders of each region.

Due to the complex morphology and structure of calcific presentation, the single value scores developed for coronary artery calcium scoring [32, 36] precludes determining the progression of calcific deposition in AS [37, 38]. The high contrast and spatial resolution in contrast images combined with a precise calcific detection method, provides a unique opportunity to examine the regional presentation of calcific deposition in AS. In that respect, topographic distance maps were generated for both radial and longitudinal distance relative to each patient's aortic valve dimensions (annular radius and STJ height) as presented in FIGS. 5a-e. The results of 6 representative patients were arranged, grouped by sex and AS severity. Furthermore, using the anatomical 18 region mapping scheme described in FIG. 1c, this regional presentation was evaluated across the entire cohort (FIGS. 5g-j) both in terms of calcific volume and average Hounsfield intensity in each region. Furthermore, calcific volume contribution was compared between the different groups as well as between the regions for the entire cohort (FIG. 6b; Table 5; Table 6).

TABLE 5
Summary of One-way ANOVA Kruskal-Wallis post-hoc analysis (N = 178). Results of
regional volume post-hoc pairwise comparisons between categories comparing sex and severity
effect on regional calcific distribution, p-values reported to 3 significant figures

| | One-Way ANOVA (Non-parametric) | | | | | | | | | | | |
|---|---|---|---|---|---|---|---|---|---|---|---|---|
| | Dwass-Steel-Critchlow-Fligner pairwise comparisons | | | | | | | | | | | |
| Region | F-NS | F-S | M-NS | M-S | F-S | M-S | F-NS | M-S | F-S | M-NS | F-NS | M-NS |

| | | | | | | |
|---|---|---|---|---|---|---|
| R1 | 0.197 | 0.316 | 0.002 | <.001 | 0.836 | 0.102 |
| R2 | 0.242 | 0.025 | 0.046 | <.001 | 0.886 | 0.757 |
| R3 | 0.172 | 0.363 | 0.054 | <.001 | 0.976 | 0.105 |
| R4 | 0.006 | 0.036 | <.001 | <.001 | 0.681 | 0.003 |
| R5 | <.001 | 0.126 | 0.006 | <.001 | 0.982 | <.001 |
| R6 | 0.003 | 0.136 | 0.006 | <.001 | 0.887 | <.001 |
| R7 | 0.01 | <.001 | 0.003 | <.001 | 0.121 | 0.912 |
| R8 | 0.008 | 0.173 | 0.113 | <.001 | 0.991 | 0.203 |
| R9 | 0.096 | 0.261 | 0.023 | <.001 | 0.947 | 0.113 |
| R10 | 0.052 | 0.002 | <.001 | <.001 | 0.949 | 0.264 |
| R11 | 0.002 | 0.03 | 0.041 | <.001 | 0.603 | 0.356 |
| R12 | 0.003 | 0.008 | <.001 | <.001 | 0.874 | <.001 |
| R13 | 0.007 | 0.027 | 0.005 | <.001 | 1 | 0.068 |
| R14 | 0.057 | 0.099 | 0.161 | 0.001 | 0.826 | 0.447 |
| R15 | 0.002 | 0.023 | 0.001 | <.001 | 0.979 | 0.026 |
| R16 | 0.271 | 0.054 | 0.107 | 0.003 | 0.816 | 0.855 |
| R17 | 0.392 | 0.298 | 0.969 | 0.179 | 0.515 | 0.994 |
| R18 | 0.242 | 0.987 | 0.997 | 0.345 | 0.955 | 0.688 |

TABLE 6
Summary of paired sample t-tests Wilcoxon rank (N = 178). Entire cohort was used
in the analysis, pairs of adjacent regions in each leaflet cusp were used to
evaluate statistical differences in regional volume across the valve surface

| | | | | | Standard |
|---|---|---|---|---|---|
| | | | | Mean | Error |
| Paired Samples T-Test (Wilcoxon W) | | W | p-value | Difference | Difference |
| Coaptation Zone (NCC)- | Belly Left (NCC)-R4 | 184 | <.001 | −52.182 | 4.33 |
| R1 | Belly Right (NCC)-R5 | 339 | <.001 | −42.766 | 4.77 |
| Belly Left (NCC)-R4 | Attachment Left (NCC)-R10 | 8985 | 0.056 | 6.946 | 4.04 |
| | Attachment Mid (NCC)-R11 | 10466 | <.001 | 15.868 | 4.85 |
| Belly Right (NCC)-R5 | Attachment Mid (NCC)-R11 | 9659 | 0.009 | 9.758 | 4.68 |
| | Attachment Right (NCC)-R12 | 9077 | 0.079 | 4.172 | 3.99 |
| Coaptation Zone (RCC)- | Belly Posterior (RCC)-R6 | 734 | <.001 | −33.118 | 3.57 |
| R2 | Belly Anterior (RCC)-R7 | 739 | <.001 | −30.991 | 3.43 |
| Belly Posterior (RCC)- | Attachment Posterior (RCC)-R13 | 8640 | 0.264 | 2.969 | 4.58 |
| R6 | Attachment Mid (RCC)-R14 | 12006 | <.001 | 16.889 | 3.84 |
| Belly Anterior (RCC)-R7 | Attachment Mid (RCC)-R14 | 11386 | <.001 | 13.096 | 3.79 |
| | Attachment Anterior (RCC)-R15 | 9823 | 0.007 | 7.308 | 3.59 |
| Coaptation Zone (LCC)- | Belly Anterior (LCC)-R8 | 1400 | <.001 | −24.08 | 3.15 |
| R3 | Belly Posterior (LCC)-R9 | 830 | <.001 | −37.433 | 4.08 |
| Belly Anterior (LCC)-R8 | Attachment Anterior (LCC)-R16 | 7829 | 0.848 | 0.464 | 3.87 |
| | Attachment Mid (LCC)-R17 | 8967 | 0.082 | 4.597 | 4.17 |
| Belly Posterior (LCC)- | Attachment Mid (LCC)-R17 | 10931 | <.001 | 17.933 | 4.97 |
| R9 | Attachment Posterior (LCC)-R18 | 11546 | <.001 | 20.779 | 4.77 |

Reference is now made to FIGS. 5a-5h and FIGS. 6a & 6b. FIGS. 6a and 6b show examples of comparative analysis of calcific detection methods and regional distribution of calcification across the cohort (N=178). FIG. 6a—Spearman's correlation heatmap between all contrast calcific volume scores and non-contrast volume and Agatston scores. FIG. 6b—Radar plot with categories grouped by sex and AS severity showing regional median volume spread in each region defined in FIG. 1c.

In terms of the regional presentation of calcification for each cusp (FIG. 5), four primary patterns of calcific distribution were observed as follows:
1. Severe: Maximal calcific deposition in near the belly region of all leaflets followed by root attachment edges with significantly lower calcification near the coaptation zones (FIGS. 5a-d; FIGS. 5g, h; Table 6). In males, the amount of calcification is significantly higher across most regions (FIG. 6b, Table 5). In females (FIG. 5b, d; FIG. 5h) a lower calcific progression rate presents calcific arcs primarily near free edges of the leaflets that have not yet developed in rings at the fixed edge (FIG. 6b; Table 5).
2. Non-severe: Maximal calcific deposition near sites of root attachment and belly, prominent near the non and left coronary cusps (FIGS. 5e, f; FIGS. 5i, j). At this stage calcification primarily presents as disconnected deposits with males presenting significantly higher calcific deposition near leaflet attachment edges between the non and right coronary leaflets than females (FIG. 5f; FIG. 5j; FIG. 6b; Table 5).

Overall inter-region comparison (FIG. 6b; Table 6) shows a radial spoke pattern of calcific progression that is clustered around the belly region of the leaflets, followed by the root attachment at both sides of the leaflets and significantly decreasing near the coaptation zone. In terms scale the non-coronary leaflet experiences the most calcific thickening followed by the right and then left coronary leaflet.

It may be noted that current in-vivo quantitative description of regional calcification is limited [13] and most reported studies relied on excised tissue analysis [37] or biomechanical simulated models [39, 40]. The described patient-specific geometric model was designed to capture the local pattern of calcific deposition. The described results indicate that although calcific progression in each leaflet follows a particular pattern that agrees with reported literature [13, 37, 39, 40], the rate of calcific progression is clearly asymmetric across the different leaflets. Furthermore, females experience significantly less calcific deposition within those patterns despite experiencing similar degrees of valve obstruction. This observation concurs with reported data [18, 41, 42], which may add further evidence that calcific thickening alone is not enough to cause significant valve obstruction and subsequent clinical symptoms of AS.

### Discussion Based on the Example Results

AS is generally characterized as a gradual decrease of the valve orifice area leading to left ventricular outflow tract (LVOT) obstruction and later, left ventricular (LV) hypertrophy due to an increased afterload. There are two primary pathways for AS development, calcific deposition, and rheumatic heart disease. Globally, calcific AS accounts for the majority of AS pathology particularly in developed countries with prevalence increasing with age [6, 8, 9]. Accurate quantification of calcific burden is therefore critical in the AS diagnostic process, particularly in situations when standard diagnostic measures (e.g., echocardiography) are inconclusive [32].

Techniques using contrast-enhanced imaging [17-22], which are based on luminal attenuation or a fixed HU threshold, have been reported to show promise in terms of ease of use and reproducibility. Despite their benefits, these techniques may underestimate calcification in the leaflets, especially if image noise or calcific deposits in the sino-tubular junction (STJ) plane are present. Additionally, these methods may be sensitive interscan, inter-device and cohort selection, which may explain the wide variation of values used for calcium thresholding [17-22]. The disclosed systems and methods can dynamically adjust the threshold based on local image features. Finally, using high detail contrast imaging, presents a potential to move beyond the bulk volume or Agatston scores [18, 43]. New markers based on the regional patterns of calcification could be a valuable add-on to existing CT-TAVR guidance protocols, given that in current CT-TAVR [12] assessment, AS severity is already confirmed and valvular calcification is only described qualitatively to plan interventions. Another motivation for developing new markers for measuring calcific burden may be the strong evidence that shape, position, and density distribution of calcification are all closely related to peri-procedural outcomes [12, 13, 44]. Disclosed FPR method provides an attenuation stable calcific detection threshold

A key advantage of the FPR method can be that by considering a shape overlap measure in guiding the optimization for calcium threshold detection, the method can better adapt to general fluctuations in the intraluminal contrast. Therefore, it can work equally well in both high and low calcific densities. The FPR method (FIG. 2) may rely on the automatic estimation of the false positive rate of detected contrast material [21] to guide the detection of an optimal threshold of calcific regions. This is based on the idea that the detection of scattered calcific lesions can be guided by the more reliable estimation of the larger aortic root model with well-defined boundaries. Indeed, the direct evaluation of AS scoring is currently based on non-contrast images [12, 32]. The volume scores derived using the disclosed method had the strongest correlation with standardized non-contrast calcific scores (FIG. 6a) and can further demonstrate the limitations of fixed and luminal attenuation methods, particularly in either under or overestimation of calcific content at high or low calcific concentrations, respectively (FIGS. 4c-f). This may be particularly important in improving the reproducibility of calcification assessment using contrast images in large, randomized cohorts, especially in females. Despite exhibiting significantly lower calcific deposition in compared to males (FIG. 5), females still present with symptoms of severe AS [41]. In addition, a more precise calcific detection method can have added prognostic use for TAVR planning [12] by determining the regional effect of calcific deposition in periprocedural scenarios [12, 13, 44](e.g., paravalvular leakage, left-bundle branch block).

A precise calcium detection method can be necessary to accurately evaluate regional calcific distribution and hence understand the more likely pattern of progression. Furthermore, once a calcific threshold is identified, the HU intensity distribution specific to calcific regions can be evaluated (FIG. 1b; FIGS. 5g-i). With higher intensity corresponding to denser calcific concentrations, this may also correspond to earlier vs later calcific deposition [40]. From one aspect, diffuse regions may be attributed to late-stage leaflet fibrosis [18]. Another aspect of calcification intensity grading is the possibility of predicting leaflet mobility, given that the pattern and rate of calcific progression affects the tissue material properties inducing impairment to physiological function [37-40].

Volume Scores for Patients with AS in Contrast-Enhanced Images are Significantly Lower than Those Using Non-Contrast Images

Previous reports of volume scores using contrast images [19-22] were considerably lower compared to expected volume scores in AS [23, 32]. This apparent underestimation, even when using different techniques, was not sufficiently discussed in the literature. Quantifying this bias can have important implications for both the diagnostic and prognostic use cases of contrast CT imaging. Indeed, quantifying this relative error in measurement can decrease uncertainty in deriving sex-specific thresholds for AS severity determination. In terms of the comparing contrast and non-contrast calcific estimation, a close qualitative agreement with detected calcification, both in terms of location relative to aortic leaflet surface and pattern of distribution was observed (FIG. 3). Additionally, a significant correlation in volume between both non-contrast and contrast images was demonstrated (FIG. 6a). Despite these agreements, the total volume scores for contrast images seem to be significantly underestimated compared to those for non-contrast images (FIG. 3; FIG. 4b; Table 4). Potential reasons for this disagreement may be as follows [46, 47]. The non-contrast Agatston protocol uses a lower axial resolution (3 vs 0.625 mm), which may cause an overestimation of large calcific nodules and underestimation of smaller deposits. This is due to the increased slice thickness via the partial volume effect [48]. This error would be nonlinear and sensitive to local calcium densities and orientation, which can be observed in the described experiments (FIG. 3b; FIG. 3h; FIGS. 4b-f). And this can also explain observed underestimation of calcium scores when using the “en-face” reconstructed view [49]. Secondly, dense calcific regions with large HU values may produce blooming artifacts [47] which could further compound the volume error with a higher slice thickness. Both factors may imply a more accurate volume measurement using contrast images with lower slice thickness and contrast differentiation between the tissues.

### Varied Patterns of Calcific Distribution can be Detected and Quantified Using Contrast CT

To better assess calcification in various regions of the cardiovascular system, new quantitative criteria [45] were suggested to go beyond previously used calcification assessment methods [36] which only relied on the quantity of calcification. These markers may be especially important in the context of TAVR planning since different factors can influence the severity of various complications during and post-operation such as the location, quantity, and type of the calcific deposits [12, 13, 44]. The described geometrical mapping framework can quantify the topology of the calcification in the aortic valve based on the common anatomical definitions of a tricuspid aortic valve [50] and can focus on the leaflet region of the valve to provide an in-vivo quantitative description of the pattern of calcific progression in AS.

Using the disclosed systems and methods, qualitative and quantitative patterns of calcific distribution were demonstrated that may closely match those demonstrated in ex-vivo studies by Thubrikar et al. [37] and those posited in biomechanical simulated models [38-40]. The described experiments can chiefly entail the following: calcification advances primarily from the high stress regions of the valve leaflets which form arcs and eventually rings covering the leaflet's surface. Among the four distinctive patterns previously described [38-40], it was observed that the primary pattern of calcific arcs forming between the two attachment edges of each leaflet along the belly seems to dominate across sex and severity categories (FIGS. 5g-j; FIG. 6b). In severe tricuspid cases, calcific nodules form arcs connecting the root attachment points and eventually form fully connected rings that conform to the free and fixed edge of the leaflets from both sides (FIG. 5). In contrast, non-severe AS cases have calcification that is less developed and concentrated near the coaptation between the non and left coronary leaflets (FIG. 5). It was also demonstrated that this pattern is presented in both males and females with females experiencing a significantly lower amount across all regions (FIG. 5; FIG. 6). Differences in aortic valve size do not sufficiently explain this wide discrepancy (Table 4). This finding provides further evidence that biomechanics of functional impairment in females are not dominated by calcific leaflet thickening and it seems that leaflet fibrosis could play a much bigger role in worsening clinical symptoms for females with AS [18, 41, 42]. In addition, it was shown that asymmetry in cusp sizes and shapes [51] leads to analogous asymmetry in quantity and pattern of calcific distribution across the valve. Regarding regional volume scores, a relative increase in non-coronary cusp calcification was shown (Table 4), which agrees with previous experiments using similar patient cohorts and image modalities [13]. The relatively longer length of the NCC35 along with the direction of calcific progression can explain the discrepancy in volume. This observation could have important implications for post-procedural complications due to the proximity of NCC calcification to the ventricular conduction system [12, 13, 44]. In summary, accurate quantification of the asymmetry in calcific deposits may be important to avoid various procedural complications if related to the positioning and expansion of the implant [13, 16, 20].

The described results may shed further light on the relationship between calcific leaflet thickening, subsequent valve obstruction and presentation of clinical symptoms [52]. A better understanding of the biomechanical causes of impaired leaflet mobility may provide an opportunity to decide on optimal intervention times and further staging criteria [53].

### Limitations of the Described Example Results

The preliminary findings described herein are based on an observational cohort of patients. The non-consecutive cohort was chosen to evaluate and verify the disclosed methods in a balanced distribution of sex and AS severity which may not be reflected in a larger population study. Further investigation in a multi-center setting may be needed to better judge the effectiveness of the disclosed method in more clinical contexts as well as to better judge interscan and inter-device variability. Additionally, some of the proposed metrics for calcification assessment may need to be further analyzed in the context of peri-procedural outcomes in TAVR/TAVI. Additional indices based on the disclosed descriptors may provide a stronger prognostic impact in relation with implantable device size, type, and expansion behavior.

### Conclusions Based on the Described Example Results

Calcific AS is the most common valvular disease, with high mortality rate once symptoms are presented. AVR intervention is currently the only treatment option in which aortic valve calcification deposited in and around the valve is an important factor for procedural risk assessment and predicting complications in TAVR. The disclosed systems and methods can be used to characterize the complex patterns of calcification at different stages of the disease. It was found that calcific progression may follow distinctive patterns for each leaflet, with differences emerging in the stage of calcific progression between the leaflets. This difference in rate of progression can also be significantly affected by sex which cannot be explained by the difference in valve sizes. In addition, a novel method is provided for calcific detection that can potentially overcome the inherent variability of contrast material effect on HU attenuation which could enable additional quantitative criteria for calcific assessment in routinely used contrast CT for TAVR planning. In summary, detailed quantitative description of the complex patterns of leaflet calcification, combined with accurate anatomical assessment of aortic valve morphology, could be critical for simultaneously monitoring the progression of AS, and determining the best treatment options, especially for asymptomatic patients who might eventually need an aortic valve replacement.

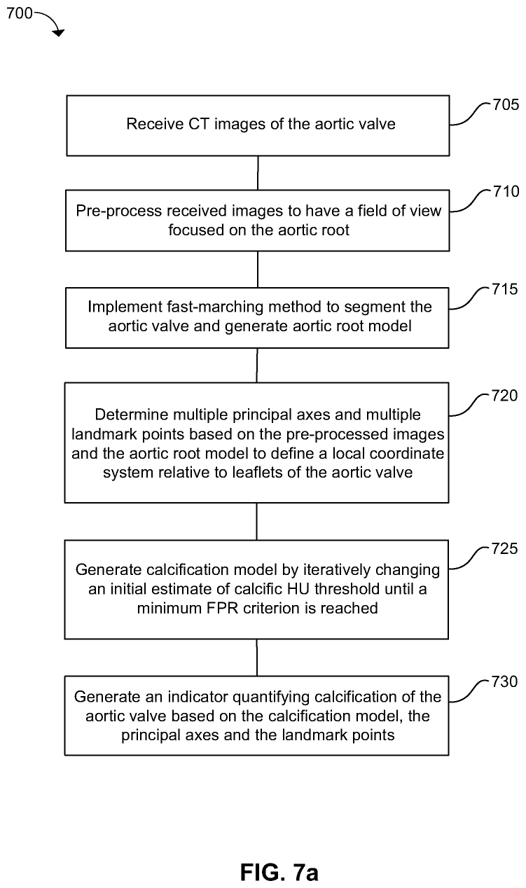

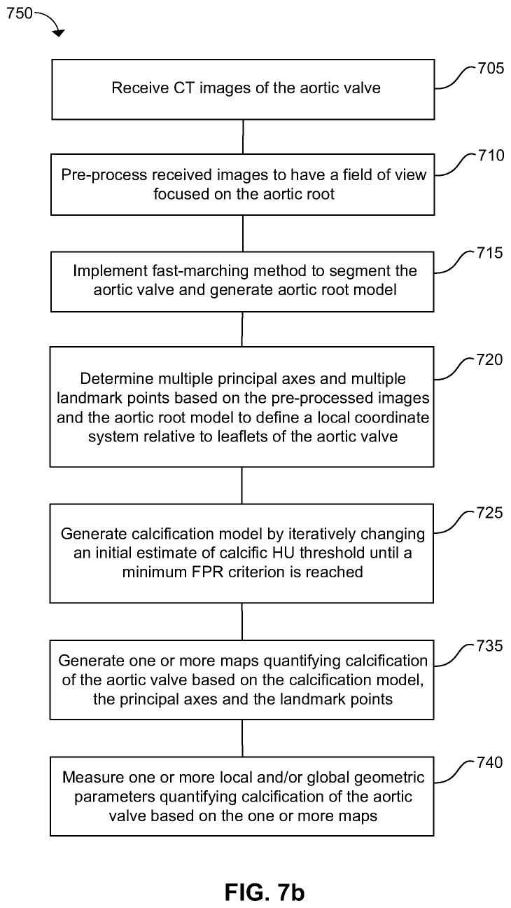

Referring next to FIG. 7a, shown therein is a method 700 for assessing calcification of an aortic valve, in accordance with one or more embodiments. Method 700 may be used for personalized and/or non-invasive cardiology of subjects. Method 700 may be used for assessing calcification of an aortic valve in any health condition, for example, healthy or unhealthy conditions, symptomatic or asymptomatic conditions etc. In one or more embodiments, the subjects may be patients with valvular diseases in both pre-intervention and post-intervention status. Method 700 may be used for monitoring, treatment planning and risk assessment in patients with valvular disease (e.g., AS) in both pre-intervention and post-intervention states. In one or more embodiments, the intervention can be TAVR and method 700 may be used for monitoring, treatment planning, risk assessment and complication/event prediction in patients with AS that will undergo TAVR.

At 705, method 700 may include receiving CT images of the aortic valve. In one or more embodiments, the CT images may be contrast-enhanced CT images.

At 710, method 700 may include pre-processing the received images to have a field of view focused on an aortic root corresponding to the aortic valve. In one or more embodiments, the received images may be pre-processed to have the field of view include an interface between the aortic valve and the LVOT, and a part of the ascending aorta after the STJ; and to have the field of view exclude any other surrounding structures. In some embodiments, the field of view may be resampled to have isotropic voxel dimensions of [0.5, 0.5, 0.5 mm]. In one or more embodiments, the field of view may be localized through a deep learning inference operation using a pretrained deep learning model.

At 715, method 700 may include implementing a fast-marching method for the pre-processed images to segment the aortic valve from surrounding tissue to generate an aortic root model. In one or more embodiments, generating the aortic root model may include performing a morphological closing operation and a smoothing recursive gaussian operation to obtain a smoothed surface. In one or more embodiments, generating the aortic root model may include a deep learning inference operation using a pretrained deep learning model.

At 720, method 700 may include determining multiple principal axes and multiple landmark points based on the pre-processed images and the aortic root model to define a local coordinate system relative to the leaflets of the aortic valve. In one or more embodiments, the multiple principal axes correspond to an anatomical short axis view, and two long axis views that are perpendicular to the “En-face” short axis view.

At 725, method 700 may include generating a calcification model based on the pre-processed images and the aortic root model by iteratively changing an initial estimate of calcific HU threshold until a minimum FPR criterion is reached. In one or more embodiments, the minimum FPR criterion may be 1%. In some embodiments, a different minimum FPR criterion may be used (e.g., 0.5% to 1%, 1% to 2% etc.). The choice of minimum FPR criterion can be based on the sensitivity and specificity required for the application. A higher FPR threshold (2-10%) can lead to higher volumes of calcific detection with higher sensitivity at the cost of lower specificity (i.e. less accuracy in differentiating between calcific and non-calcific luminal segments). A low FPR criterion (0.01-0.9%) can lead to lower volumes of calcific detection with better specificity (i.e. more precision in differentiating between calcific and non-calcific segments) with the cost of lower sensitivity in detecting the true amount of calcific pixels. In one or more embodiments, generating the calcification model may include a deep learning inference operation using a pretrained deep learning model.

At 730, method 700 may include generating an indicator quantifying calcification of the aortic valve based on the calcification model, the principal axes and the landmark points.

Referring now to FIG. 7b, shown therein is a method 750 for assessing calcification of an aortic valve, in accordance with one or more embodiments. Similar to method 700, method 750 may be used for personalized and/or non-invasive cardiology of subjects. Method 750 may be used for assessing calcification of an aortic valve in any health condition, for example, healthy or unhealthy conditions, symptomatic or asymptomatic conditions etc. In one or more embodiments, the subjects may be patients with valvular diseases in both pre-intervention and post-intervention status. Method 750 may be used for monitoring, treatment planning and risk assessment in patients with valvular disease (e.g., AS) in both pre-intervention and post-intervention states. In one or more embodiments, the intervention can be TAVR and method 750 may be used for monitoring, treatment planning, risk assessment and complication/event prediction in patients with AS that will undergo TAVR.

Steps 705-725 of method 750 may be identical to steps 705-725 of method 700. At 735, method 750 may include generating one or more maps quantifying calcification of the aortic valve based on the calcification model, the principal axes and the landmark points. In one or more embodiments, the maps quantifying calcification include one or more of a regional calcification map, a radial distance map, a longitudinal distance map and a calcification intensity map. In some embodiments, method 700 may further include generating an anatomical N region volume map and/or an anatomical N region average intensity map quantifying calcification of the leaflets of the aortic valve. The anatomical N region volume map and the anatomical N region average intensity maps may be based on the calcification model, the principal valve axes, a STJ height, an annular radius, angles between the leaflets of the aortic valve and N defining the number of discrete regions in the map/s. N may be any suitable number, for example, N may be in a range from 3 to 36 (e.g., N may be 18). The choice of the number of discrete regions N can affect the sampling rate of calcific volume in the region of interest. A large value of N (36-108) can increase the number of unique regions where calcification is sampled (i.e. it captures more information) at the cost of increasing computational time and memory requirements. A smaller value of N (3-9) can sample calcification over smaller number of unique regions which decreases computational time and memory requirements. However, with very small values of N small variations in calcific patterns may be missed entirely which would reduce the classification power of the volume/average intensity map.

At 740, method 750 may include measuring one or more local and/or global geometric parameters quantifying calcification of the aortic valve based on the one or more maps generated at 735. The calcification may be quantified in terms of global and local geometric shape descriptors based on a shape statistics algorithm which can enable automatic estimation of the physical size, the principal orientation axes, the center of mass, the boundary points, the roundness, the flatness, the elongation, the equivalent spherical radius, the equivalent ellipsoidal diameter and the fractal dimension index of each calcific lesion in the generated calcification model.

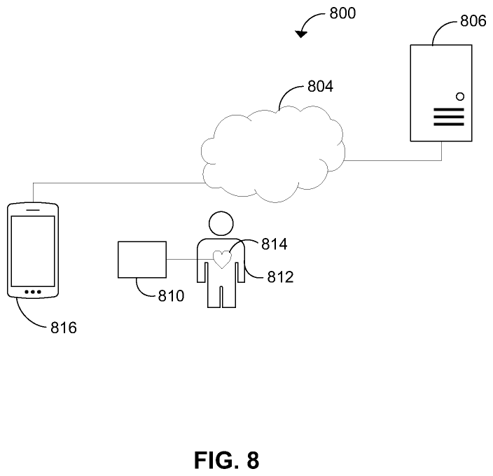

Referring next to FIG. 8, shown therein is a system 800 for assessing calcification of an aortic valve. System 800 may include one or more user devices 816, a network 804, and a server 806. Also shown is a subject 812 having a heart 814 and a CT imaging device 810.

The one or more user devices 816 may be used by an end user to access a software application (not shown), either via a web browser or locally at device 816. The software application may run at server 806 and be accessible over network 804 to the web browser at user device 816. Alternatively, the user of user device 816 may download an app from an app store such as the Google® Play Store or the Apple App Store. The user device 816 may be a desktop computer, mobile device, or laptop computer.

The user of user device 816 may be a medical professional (not shown). Optionally, the user of user device 816 may be the subject 812. Each user device 816 includes and executes a client application, such as a cardiovascular modelling application, which communicates with or otherwise receives data obtained from CT imaging device 810.

Network 804 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

CT imaging device 810 may be any suitable device capable of performing CT imaging of subject 812. In some embodiments, CT imaging device 810 may be capable of acquiring contrast-enhanced CT images of subject 812. The CT images from CT imaging device 810 may be provided to the user device 816 and/or the server 806.

The server 806 can be in network communication with the user device 816. The server 806 may have an application server and a database. The database and the application server may be provided on the same server, may be configured as virtual machines, or may be configured as containers. The server 806 may run on a cloud provider such as Amazon® Web Services (AWS®).

The server 806 may host a web application or an Application Programming Interface (API) endpoint that the user device 816 or CT imaging device 810 may interact with via network 804. The requests made to the API endpoint of server 806 may be made in a variety of different formats, such as JavaScript Object Notation (JSON) or eXtensible Markup Language (XML). The database may store acquired CT images and/or generated maps quantifying calcification of aortic valve leaflets. The database may be a Structured Query Language (SQL) such as PostgreSQL or MySQL or a not only SQL (NoSQL) database such as MongoDB.

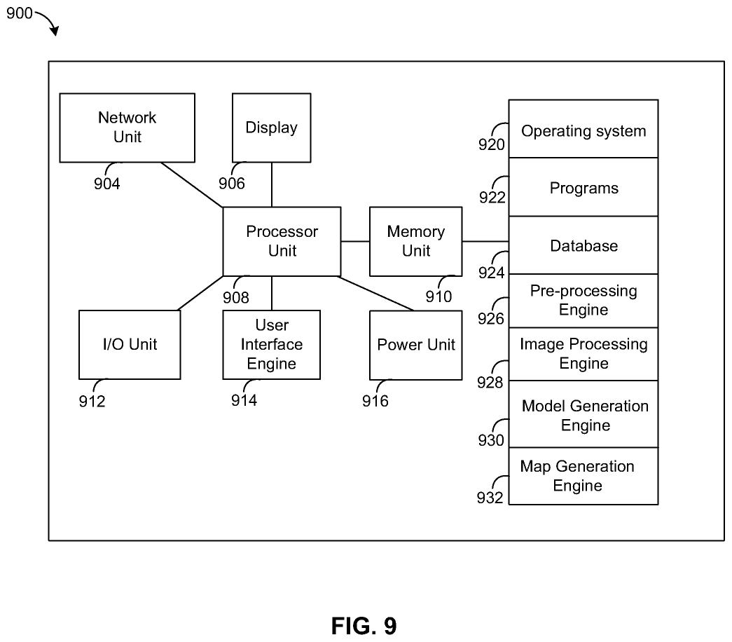

Referring now to FIG. 9, shown therein a device 900 for assessing calcification of an aortic valve, in accordance with one or more embodiments. Device 900 may, for example, provide the functionality of user device 816 of FIG. 8. In one or more embodiments, the methods described herein may be performed by device 900.

The device 900 includes one or more of a network unit 904, a display 906, a processor unit 908, a memory unit 910, I/O unit 912, a user interface engine 914, and a power unit 916.

The network unit 904 can include wired or wireless connection capabilities. The network unit 904 can include a radio that communicates utilizing CDMA, GSM, GPRS or Bluetooth protocol according to standards such as IEEE 802.11a, 802.11b, 802.11g, or 802.11n. The network unit 904 can be used by the device 900 to communicate with other devices or computers.

Network unit 904 may communicate using a wireless transceiver to transmit and receive information via a local wireless connection with a CT imaging device and/or a server. The network unit 904 may provide communications over the local wireless network using a protocol such as Bluetooth (BT) or Bluetooth Low Energy (BLE).

The display 906 may be an LED or LCD based display, and may be a touch sensitive user input device that supports gestures.

The processor unit 908 can control the operation of the device 900. The processor unit 908 can be any suitable processor, controller or digital signal processor that can provide sufficient processing power depending on the configuration, purposes and requirements of the device 900 as is known by those skilled in the art. For example, the processor unit 908 may be a high-performance general processor. In alternative embodiments, the processor unit 908 can include more than one processor with each processor being configured to perform different dedicated tasks. In alternative embodiments, it may be possible to use specialized hardware to provide some of the functions provided by the processor unit 908. For example, the processor unit 908 may include a standard processor, such as an Intel® processor or an ARM® processor.

The processor unit 908 can also execute a user interface (UI) engine 914 that is used to generate various UIs, for example, for displaying generated maps quantifying calcification of the aortic valve to a user of the device 900.

The memory unit 910 comprises software code for implementing an operating system 920, programs 922, database 924, pre-processing engine 926, image processing engine 928, model generation engine 930, and map generation engine 932.

The memory unit 910 can include RAM, ROM, one or more hard drives, one or more flash drives or some other suitable data storage elements such as disk drives, etc. The memory unit 910 can be used to store an operating system 920 and programs 922 as is commonly known by those skilled in the art.

The I/O unit 912 can include at least one of a mouse, a keyboard, a touch screen, a thumbwheel, a track-pad, a track-ball, a card-reader, voice recognition software and the like again depending on the particular implementation of the device 900. In some cases, some of these components can be integrated with one another.

The user interface engine 914 may be configured to generate interfaces for users to configure CT scans, connect to the CT imaging device and/or a server storing acquired CT images, view maps quantifying calcification of the aortic valve etc. The various interfaces generated by the user interface engine 914 may be displayed to the user on display 906.

The power unit 916 can be any suitable power source that provides power to the device 900 such as a power adaptor or a rechargeable battery pack depending on the implementation of the device 900 as is known by those skilled in the art.

The operating system 920 may provide various basic operational processes for the device 900. For example, the operating system 920 may be a mobile operating system such as Google® Android® operating system, or Apple® iOS® operating system, or another operating system.

The programs 922 include various user programs so that a user can interact with the device 900 to perform various functions such as, but not limited to, receiving CT images, viewing maps quantifying calcification of the aortic valve etc.

The database 924 may be a database for storing CT images, model parameters and generated maps quantifying calcification of the aortic valve of one or more subjects.

The pre-processing engine 926 may process received CT images to have a field of view focused on an aortic root corresponding to the aortic valve. In one or more embodiments, the pre-processing engine 926 may process received images to have the field of view include an interface between the aortic valve and a LVOT, and a part of the ascending aorta after a STJ; and to have the field of view exclude any other surrounding structures.

The model generation engine 930 may generate an aortic root model and/or a calcification model. For example, the model generation engine 930 may implement a fast-marching method for the pre-processed images to segment the aortic valve from surrounding tissue to generate an aortic root model. The model generation engine 930 may generating the calcification model based on the pre-processed images and the aortic root model by iteratively changing an initial estimate of calcific HU threshold until a minimum FPR criterion is reached.

The image processing engine 928 may receive the pre-processed images from the pre-processing engine 926. The image processing engine 928 may also receive the aortic root model from model generation engine 930. The image processing engine 928 may determine multiple principal axes and multiple landmark points that may be used to define a local coordinate system relative to the leaflets of the aortic valve.

The map generation engine 932 may generate one or more maps quantifying calcification of the aortic valve. For example, the map generation engine 932 may generate a regional calcification map, a radial distance map, a longitudinal distance map and/or a calcification intensity map. In some embodiments, the map generation engine 932 may also generate an anatomical 18 region volume map and/or an anatomical 18 region average intensity map quantifying calcification of the aortic valve.

### Example Results for Detecting Landing Zone Calcification, in the Context of Peri-Procedural Events and TAVR Complications, Using the Disclosed Systems and Methods

### Introduction

Symptomatic severe AS is the most common valvular disease requiring surgery or intervention, and prevalence continues to rise as the population ages [54-56]. Calcific degenerative disease as the leading cause of AS has a prevalence of ten percent of aged population [57, 58]. In recent years, TAVR, a minimally invasive alternative to surgical aortic valve replacement (SAVR), has become more common and is the most widely used treatment for AV stenosis [55, 59]. Unlike SAVR, during a TAVR procedure, the perivalvular area is not directly explored by the operator, and the variability in AV anatomy makes correct sizing, selecting optimal landing zone location and sufficient balloon-expansion a more challenging task [60]. This process is further complicated by the presence of calcifications on the leaflets, annulus, and left-ventricular outflow tract (LVOT). The combined calcification of these regions commonly referred to as device landing zone calcification (DLZ) [61] exhibit patient specific variations in relative density and spatial distribution, which might play a key role in prognosing short and long-term outcomes of the procedure [62, 63].

Incorrect prosthetic device sizing, over/under balloon expansion, and eccentric expansion in the TAVR landing zone are all associated with worsening outcomes leading to various degrees of paravalvular leakage/regurgitation (PVL/PVR) [64]. In addition, due to the proximity of the device with the membranous interventricular septum, cardiac conduction abnormalities due to left-bundle-branch-block (LBBB) are one of the commonest complication post-TAVR. A low implantation depth, annular/LVOT calcification and asymmetric calcific distributions are considered as important risk factors for predicting the need for pacemaker implementation post-TAVR [65-67].

In terms of procedural events, the TAVR procedure has been changed as well, with a focus on less complicated techniques that involve less frequent balloon pre-dilation (preD) of the native calcified leaflets and post-dilation (postD) of TAVR device [68]. Consequently, the TAVR procedure needs a more individualized approach, especially in light of the steadily growing usage and knowledge of TAVR as well as its adoption for low-risk patients [69]. Proper quantification of calcification patterns may be the key to such individualized approaches. Calcium deposits and their density act as mechanical obstacles that hinder stent expansion and may induce sub-optimal final configuration of the device [70, 71].

The link between calcification pattern and clinical consideration during TAVR and post-TAVR outcomes is strongly proven in single and multi-center studies [58, 72-74]. However, there appears to be a gap in the literature about the detailed assessment of calcium deposits and their impact. Contrast computed tomography angiography (CCTA) is the gold standard for TAVR planning, with its superior spatial and contrast resolution, it can provide detailed geometric and structural information about landing zone calcium deposition [12, 18]. Calcific assessment using CCTA is challenging due to variability in contrast attenuation [56], lack of standardized methodology in determining suitable calcific thresholds [64] and poorly defined relationship between contrast HU intensity and tissue density [12, 18].

In that respect, the disclosed systems and methods provide a novel semi-automatic computational framework based on CCTA images to quantify the precise geometrical and structural characteristics of calcium deposits in pre- and post-TAVR images. The example results described herein may highlight the importance of considering the relative density in addition to the spatial location of the deposition representing marked differences in patients with adverse outcome. It does not appear that there has been any reported study that sheds light on the causal link between the relative density and 3D spatial distribution of calcium deposits with the procedural events and post-TAVR complications.

### Methodology

### Study Population

The study consisted of patients who underwent TAVR with a balloon-expandable Edwards Sapien 3, Edwards Sapien 3 Ultra, or Edwards Sapien CT THV (Edwards Lifesciences, Irvine, California) from two medical centers (Hamilton General Hospital, Ontario, Canada, St. Paul's Hospital, Vancouver, Canada) between 2020 and 2022. Patients included in the analysis were required to have undergone gated contrast CT for TAVR planning and 2D Doppler echocardiography assessment before and after intervention. After a review of 251 patients evaluated for TAVR, 136 patients were included in the study. Patients with bicuspid valve morphology, self-expandable devices, those treated for surgical aortic bioprosthesis degeneration (i.e., valve-in-valve) and those with unsuccessful devices as per VARC-3 (Valve Academic Research Consortium 3) criteria were excluded [75]. No patients were excluded based on image quality. The procedural access route, THV type and sizing was determined by the local heart teams on basis of annular area and qualitative calcification grade as per recommended SCCT guidelines [12].

Among the study cohort 37 patients underwent CT follow-up from 9-12 months after intervention. Follow-up CT was based on choosing patients with higher risks of adverse events. Waiver of informed consent and data transfer was approved by the Institutional Review Boards of the respective institutions (iREB). Data collection and clinical measurements were performed by operators blinded to the objectives and contents of this study. Standard measurements were performed per relevant guidelines and regulations including guidelines of the American College of Cardiology and American Heart Association. Demographic and peri-procedural notes were collected from the patients' medical records (see Table 7 for patient characteristics).

TABLE 7
Baseline patients characteristics of study cohort (N = 136).
Baseline characteristics and procedural information presented
as median [25th-75th percentile] for continuous variables
and count (%) for categorical variables.

| | Category | Number (Percentage) |
|---|---|---|
| Age (years)-Median | | 82 [76-85] |
| [Interquartile range] | | |
| Sex-n(%) | Female | 56 (41.2%) |
| | Male | 80 (58.8%) |
| Transcatheter aortic valve Size- | 20 mm | 5 (3.7%) |
| n(%) | 23 mm | 30 (22.4%) |
| | 26 mm | 55 (41%) |
| | 29 mm | 44 (32.8%) |
| Transcatheter aortic valve | Sapien 3 | 68 (50.7%) |
| Type-n(%)ₙ₌₁₃₄ | Sapien Ultra | 43 (32.8%) |
| | Sapien XT | 22 (16.4%) |
| NYHA Class-n(%) | I | 4 (3%) |
| | II | 59 (43.4%) |
| | III | 63 (46.3%) |
| | IV | 10 (7.4%) |
| Coronary artery disease-n(%) | Yes | 69 (52.7%) |
| Hypertension-n(%) | Yes | 116 (89.2%) |
| Type 2 Diabetes mellitus-n(%) | Yes | 51 (41.5%) |
| Dyslipidemia-n(%) | Yes | 99 (78.6%) |
| Atrial fibrillation-n(%) | Yes | 45 (36.9%) |

### CT Acquisition

CT imaging was performed on a GE 320 detector Revolution scanner (General Electric, Milwaukee, United States) or a Siemens 64 detector dual (Somatom Definition Flash, Siemens, Erlangen, Germany) with a standard TAVR CT protocol (Tube voltage 120 kV, tube current modulated from 150 to 725 mA based on patient size. Modes of the acquisition were assumed volume scan for GE revolution and helical for the Siemens Flash. Prospectively ECG gated non-contrast CT was performed in a sequential mode at 60%-80% of the RR interval using a slice thickness of 3 mm. Contrast CT images were acquired using retrospective gating without tube current modulation of the entire cardiac cycle. Contrast injection rates depended on patients' size, ranging from 4.5 ml/see to 6.5 ml/sec. Timing of contrast injection used bolus-tracking technique. A single bolus of contrast was administered to capture the cardiac structures and peripheral vasculature. The amount of contrast used was based on the patient's estimated glomerular filtration rate (eGFR). Patients with an eGFR of less than 30 mL/min received 60 mL of contrast, whereas those with an eGFR of 30 mL/min or greater received 100 mL of contrast.

### Image Analysis

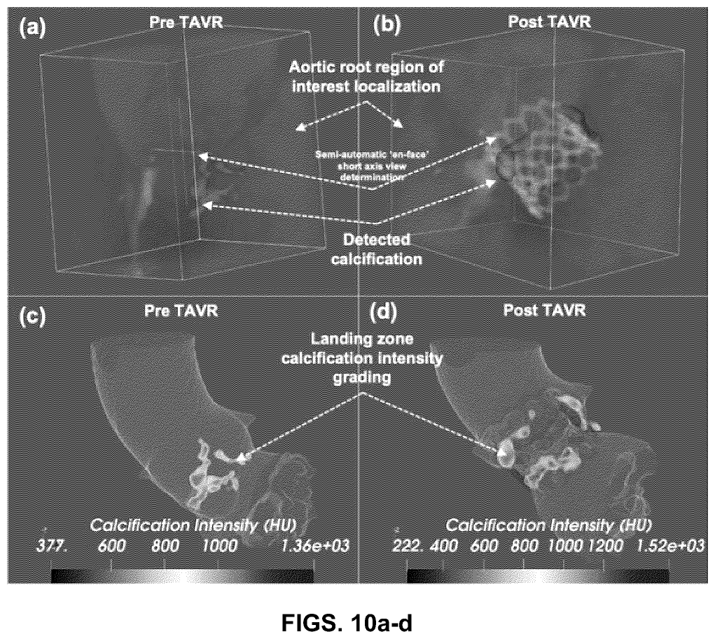

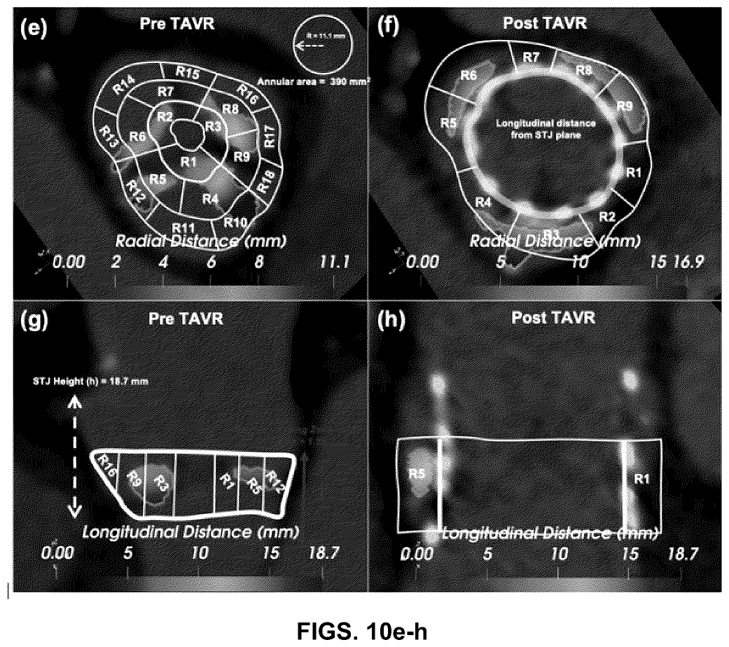

Reference is now made to FIGS. 10(a)-(h) showing example visual representations of the described computational framework for the assessment of calcification pattern and structure in contrast enhanced CT images for TAVR framework. FIGS. 10(a-b): Initial localization of the region of interest followed by automatic detection of calcification (red outline) based on optimization of the false positive rate between aortic root lumen and calcific segments. In the post intervention CT image, a distance threshold based on the device nominal expansion diameter is used to separate the stent from surrounding calcification. FIGS. 10(c-d): Detected calcific segments overlaid on the surface of the aortic root segmentation (white outline), the device stent (blue outline) colored by local HU intensity using an intensity range of minimum-maximum calcific threshold. FIGS. 10(e-f): 2D “en-face” short axis view near the annular plane showing radial calcification distance to annular center using a range of [0-annular-radius]. In the pre TAVR CT an 18-region cylindrical coordinate map is used to model the regional contribution of different regions in the landing zone. In the post TAVR CT, due to the expansion and compression of the native leaflets, a 9-region map is used to model the regional contribution in terms of volume and average density. FIGS. 10(g-h): 2D long-axis view showing the left and non-coronary cusps showing longitudinal calcification distance from STJ height to annular plane using a range of [0-STJ height]. Region subdivisions (R1-R18; R1-R9) are based on parametric coordinates in cylindrical coordinate system, normalized by patient specific aortic root dimensional measurements (STJ height h, annular area derived radius r and angles θ between the interleaflet edges measured from the “en-face” short axis view of the valve.

The disclosed method can semi-automatically find a threshold to minimize the false positive rate (FPR), defined as the ratio of falsely labeled calcific pixels to the total number of calcific pixels (FIGS. 10a, b). The geometric and relative concentration of calcification can also be described via intensity as well as radial and longitudinal maps (FIGS. 10e, f, g, h). The developed computational framework may require the field of view to initially be localized to the aortic valve based on the standard assessment guidelines for TAVR planning (FIGS. 10a, b). Subsequently, density and topographic maps may be generated of both radial and longitudinal measurements for the detected calcific regions (FIGS. 10c, d, e, g, h). The disclosed method also includes an anatomical region mapping scheme (FIGS. 10e, f) based on standardized measurements of height to sino-tubular-junction, annular area derived radius, and angles of the interleaflet triangle (FIGS. 10e, g). These measurements were used as relative distance thresholds in order to quantify regional calcific volume and average Hounsfield intensity for each patient (FIGS. 10c, d, e, f). For the post-TAVR CT images, the disclosed method can be used to detect calcification around the device, with an added step of a radial distance threshold using the nominal expansion diameter to separate the calcification from the metallic stent.

### Validation/Statistical Analysis

The statistical tests were performed using Jamovi v.1.8. Summaries of the variables and tests performed in (Table 8; Table 9) are outlined as follows. Correlations, and comparisons between the variables was performed using Spearman's rank correlation test, Wilcoxon rank paired t-test, Binomial and multinomial logistic regression was used to evaluate combined effect of the dependent variables. Receiver operator characteristic curves were used to evaluate the classification power of selected parameters (Table 9). All values were reported as median [25^{th}-75^{th} percentile]. Finally, interobserver variability was measured using the intraclass correlation coefficient (ICC; two-way random agreement) for a sub cohort of 56 patients. Statistical significance was considered when the p-value was less than 0.05.

TABLE 8
Summarized statistics (N = 136). Grouped by PVL: Paravalvular leakage, LBBB: Left-
bundle-branch-block, PreD: Baloon pre-dilation and PostD: Baloon post-dilation.
Parameter values presented as median [25th-75th percentile] for each subgroup

| Parameter | PVL_{(n = 27)} | LBBB_{(n = 14)} | PreD_{(n = 13)} | PostD_{(n = 25)} | Control_{(n = 68)} |
|---|---|---|---|---|---|
| Device | 441[298-920] | 542[310-1554] | 972[658-1404] | 432[272-546] | 583[378-860] |
| landing | | | | | |
| zone | | | | | |
| calcific | | | | | |
| volume | | | | | |
| (mm³) | | | | | |
| Indexed | 80.2[69.2-183.3] | 132[82-280] | 181[143-242] | 97.2[64-135] | 121[69.2-172] |
| device | | | | | |
| landing | | | | | |
| zone | | | | | |
| calcific | | | | | |
| volume | | | | | |
| (mm³/cm²) | | | | | |
| Calcific | 423[371-463] | 420[390-552] | 439[391-506] | 513[406-587] | 456[410-517] |
| Threshold | | | | | |
| (HU) | | | | | |
| Mean | 764[652-796] | 793[740-849] | 785[732-846] | 839[712-916] | 801[730-856] |
| Calcific | | | | | |
| Intensity | | | | | |
| (HU) | | | | | |
| Device | 222[119-377] | 261[132-768] | 391[294-717] | 191[118-314] | 269[160-406] |
| landing | | | | | |
| zone | | | | | |
| calcific | | | | | |
| volume- | | | | | |
| NCC | | | | | |
| (mm³) | | | | | |
| Device | 103[49-372] | 172[76-443] | 299[232-429] | 98[70-202] | 150[67.2-261] |
| landing | | | | | |
| zone | | | | | |
| calcific | | | | | |
| volume- | | | | | |
| RCC | | | | | |
| (mm³) | | | | | |
| Device | 161[63-316] | 138[94.3-244] | 151[116-256] | 102[53-203]] | 167[91.6-265] |
| landing | | | | | |
| zone | | | | | |
| calcific | | | | | |
| volume- | | | | | |
| LCC | | | | | |
| (mm³) | | | | | |
| Annular | 5.2[4.3-6.4] | 4.4[3.8-5.6] | 4.9[4.3-6.0] | 4.4[4.0-5.2] | 5.1[4.4-5.5] |
| area | | | | | |
| (cm²) | | | | | |
| Annular | 12.8[11.7-14.2] | 11.9[10.9- | 12.5[11.6- | 11.9[11.3- | 12.6[11.8- |
| radius | | 13.3] | 13.8] | 12.8] | 13.4] |
| (mm) | | | | | |
| STJ | 21.1[18.7-22.6] | 21.1[20-23.2] | 22.8[19.8- | 20.5[18.9- | 23.1[21.1- |
| Height | | | 27.1] | 23.3] | 25.5] |
| (mm) | | | | | |

TABLE 9
Summary of binomial logistic regression results. Results of binomial
logistic regression results comparing PVL: Paravalvular leakage,
LBBB: Left-bundle-branch-block, PreD: Baloon pre-dilation and PostD:
Baloon post-dilation against control. Regional volume and average
intensity values were used as dependent variables

| | | Cutoff | Sensitivity | Specificity | | |
|---|---|---|---|---|---|---|
| Model_{df=36} | AUC | (ratio) | (%) | (%) | R² | p value |
| PVL | 0.92 | 0.25 | 84.4 | 85.2 | 0.558 | 0.009 |
| LBBB | 0.946 | 0.1 | 82 | 78.6 | 0.612 | 0.021 |
| PreD | 0.953 | 0.1 | 83.7 | 84.6 | 0.615 | 0.162 |
| PostD | 0.916 | 0.22 | 85.6 | 84% | 0.568 | 0.01 |

### Example Results

The Contrast-enhanced CT images are frequently used for TAVR procedural planning and risk assessment which involves determining the landing zone for the valve including the LVOT, aortic annulus and valve cusps [12]. The presence of severe calcification, particularly in the annulus/LVOT is associated with an increased risk of paravalvular leakage (PVL), annular rupture, and atrio-ventricular block (LBBB) [12, 44]. Typically, calcification is exclusively assessed via a qualitative grading scheme with grades mild, moderate, and severe depending on morphology, extent and relation to aortic valve cups [12]. The location, depth and shape of these calcific lesions varies significantly across patients referred for TAVR [12, 13]. Any visual abnormalities found regarding these variables should be reported during the procedure planning phase [18].

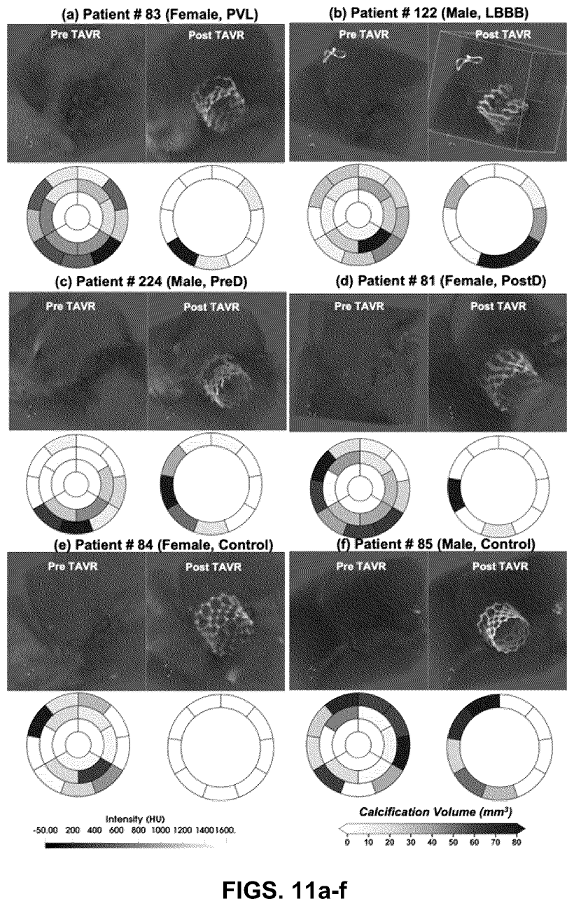

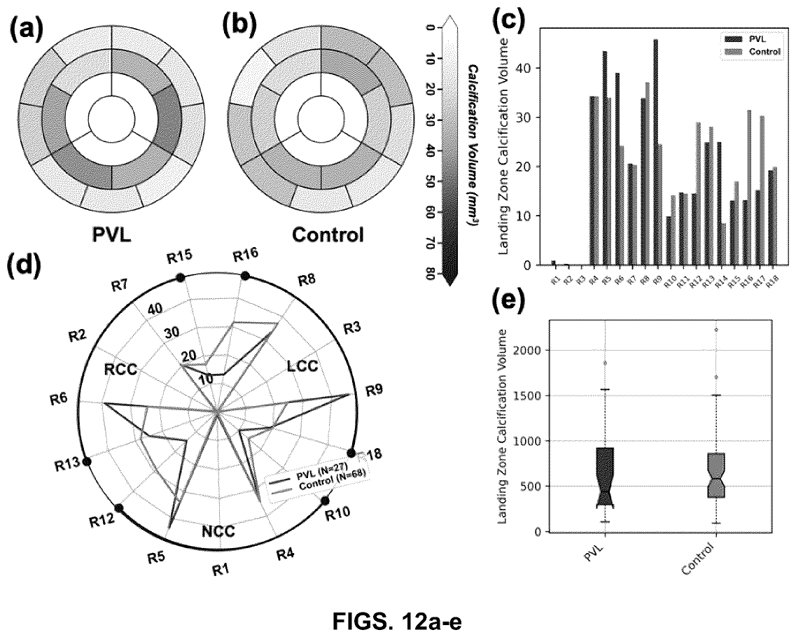

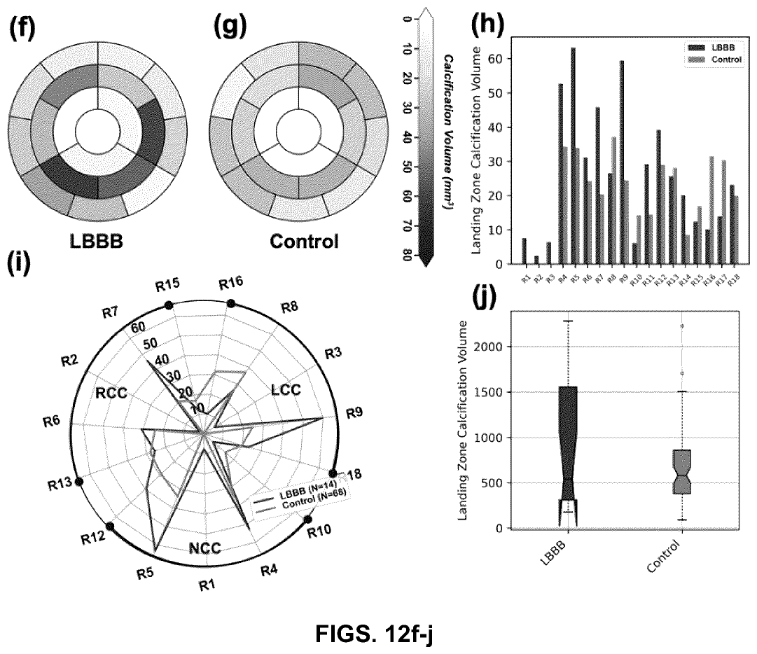

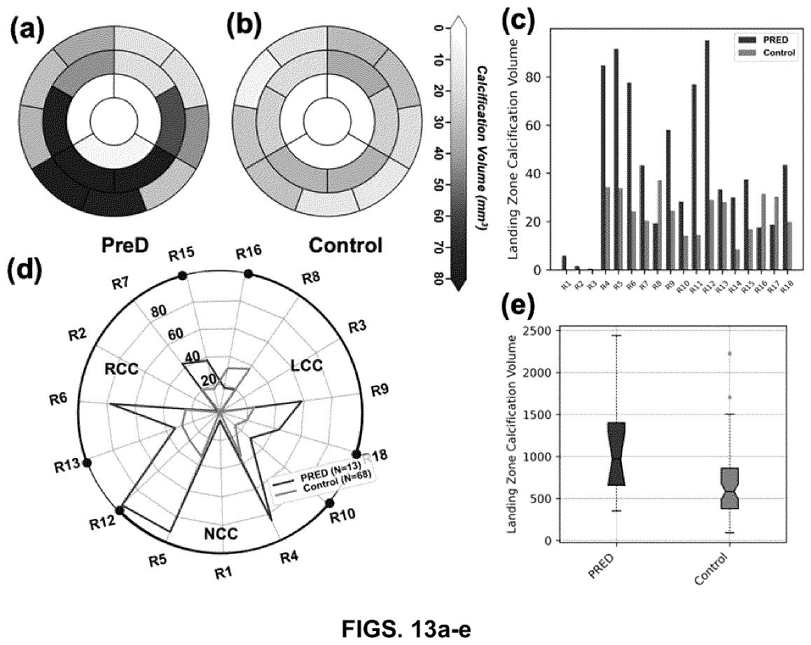

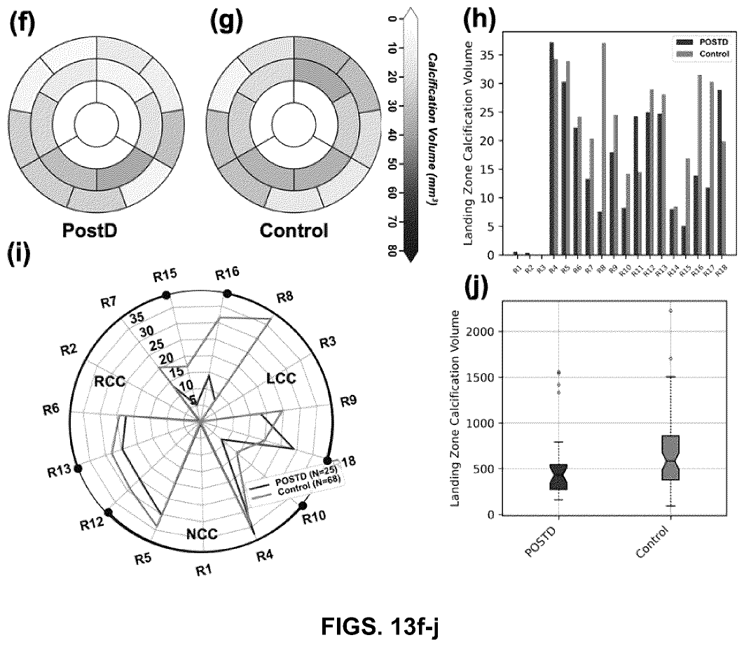

In that respect, topographic maps were generated for both radial and longitudinal distances relative to each patient's aortic valve dimensions (annular radius and STJ height) as presented in FIG. 10. Furthermore, using the anatomical 18 region mapping scheme described in FIG. 10e, the pattern and structure of landing zone calcification was quantified both in terms of calcific volume and average Hounsfield intensity using different event/complication groups. The number of patients reported with at least one of the following events were grouped as follows: PVL: n=27/136 (19.8%); LBBB: n=14/136 (10%); preD: n=13/136 (9.5%); postD: n=25/136 (18.3%). A control group was used for comparison defined as patients who had none of the latter events/complications Control: n=68/136 (50%). Calcific volume contribution was compared between the representative patient of each group as well as the control cases (FIG. 11) and population-averaged volume maps in region-specific way (FIG. 12, FIG. 13).

Reference is now made to FIGS. 11 to 14. FIG. 11 shows examples of total and regional calcification in pre vs post TAVR in representative cases. FIGS. 11(a-d): Six representative patients with pre and post CT images from each of the following groups was selected: Paravalvular Leakage PVL, left-bundle-branch-block LBBB, pre-dilation PreD, post-dilation PostD and two control cases. The top-left and top-right panels show the detected calcification in the same patient before and after intervention. The bottom-left panel shows a bullseye landing zone calcification volume plot with an 18-region map pre intervention. The bottom-left panel shows a bullseye landing zone calcification volume plot with a 9 region map post intervention. For the volume maps median [IQR] values were annotated inside the borders of each region. Field of view, orientation, opacity, volume and HU intensity ranges were matched to facilitate comparison.

FIG. 12 shows examples of landing zone calcific distribution in post-procedural complications. FIGS. 12(a-b): Bullseye landing zone calcification volume plot with 18 region map comparing patients with PVL against control. FIG. 12(c): Bar plot comparing PVL vs control in terms of regional contribution of the different landing zone areas. FIG. 12(d): Radar plot with categories comparing PVL vs control in terms of regional median volume spread. FIG. 12(e): Box and whisker plot comparing total landing zone calcific volume between PVL vs control. FIGS. 12(f-g): Bullseye landing zone calcification volume plot with 18 region map comparing patients with LBBB against control. FIG. 12(h): Bar plot comparing LBBB vs control in terms of regional contribution of the different landing zone areas. FIG. 12(i): Radar plot with categories comparing LBBB vs control in terms of regional median volume spread. FIG. 120): Box and whisker plot comparing total landing zone calcific volume between LBBB vs control. PVL: Paravalvular leakage; LBBB: Left-bundle-branch-block.

FIG. 13 shows example landing zone calcific distribution in peri-procedural events. FIGS. 13(a-b): Bullseye landing zone calcification volume plot with 18 region map comparing patients with PreD against control. FIG. 13(c): Bar plot comparing PreD vs control in terms of regional contribution of the different landing zone areas. FIG. 13(d): Radar plot with categories comparing PreD vs control in terms of regional median volume spread. FIG. 13(e): Box and whisker plot comparing total landing zone calcific volume between PreD vs control. FIGS. 13(f-g): Bullseye landing zone calcification volume plot with 18 region map comparing patients with PostD against control. FIG. 13(h): Bar plot comparing PostD vs control in terms of regional contribution of the different landing zone areas. FIG. 13(i): Radar plot with categories comparing PostD vs control in terms of regional median volume spread. FIG. 13(j): Box and whisker plot comparing total landing zone calcific volume between PostD vs control. PreD: Baloon pre-dilation; PostD: Baloon post-dilation.

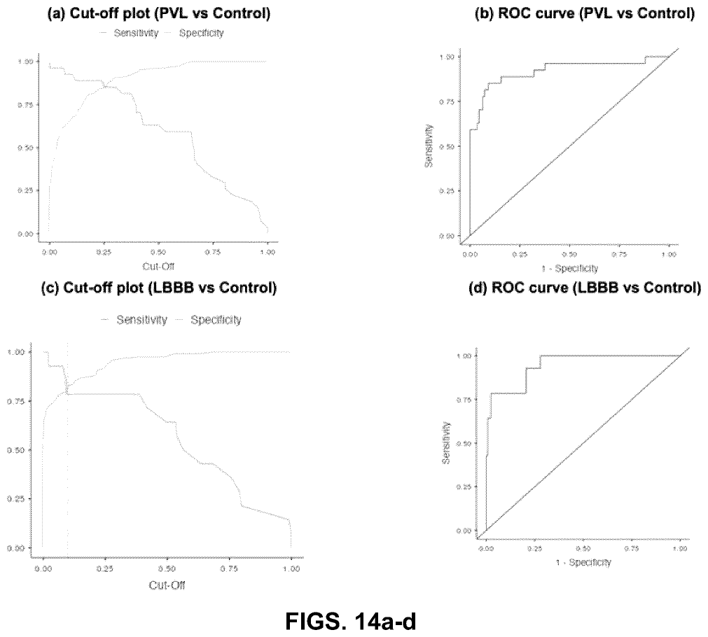

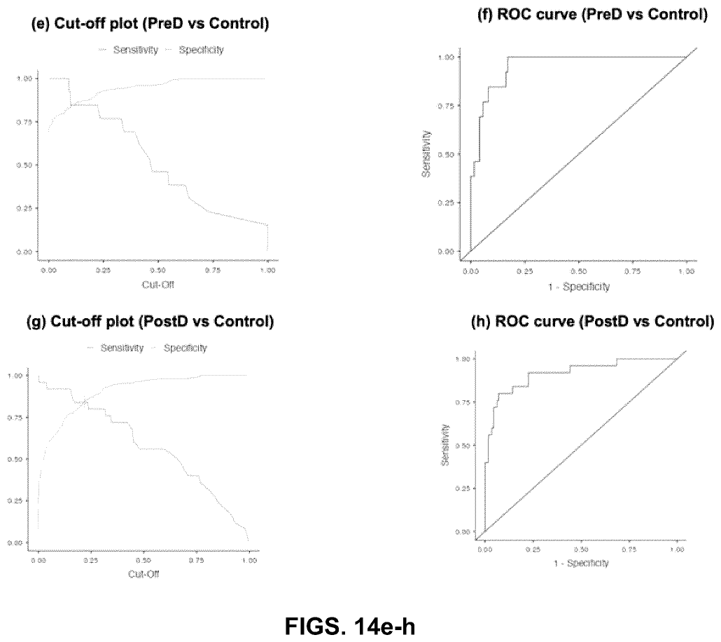

FIG. 14 shows example classification power analysis of regional landing zone calcification maps using binomial logistic regression analysis. FIG. 14(a): Cut-off plot for a 36 parameter model using 18 volume+18 average intensity values to measure classification power of the parameters in differentiation PVL from control. FIG. 14(b): Receiver operator characteristic curve ROC plot for a 36 parameter model using 18 volume+18 average intensity values to measure classification power of the parameters in differentiation PVL from control. FIG. 14(c): Cut-off plot for a 30 parameter model using 15 volume+15 average intensity values to measure classification power of the parameters in differentiation LBBB from control. FIG. 14(d): Receiver operator characteristic curve ROC plot for a 30 parameter model using 15 volume+15 average intensity values to measure classification power of the parameters in differentiation LBBB from control. FIG. 14(e): Cut-off plot for a 36 parameter model using 18 volume+18 average intensity values to measure classification power of the parameters in differentiation PreD from control. FIG. 14(f): Receiver operator characteristic curve ROC plot for a 36 parameter model using 18 volume+18 average intensity values to measure classification power of the parameters in differentiation PreD from control. FIG. 14(g): Cut-off plot for a 36 parameter model using 18 volume+18 average intensity values to measure classification power of the parameters in differentiation PostD from control. FIG. 14(h): Receiver operator characteristic curve ROC plot for a 36 parameter model using 18 volume+18 average intensity values to measure classification power of the parameters in differentiation PostD from control. PVL: Paravalvular leakage; LBBB: Left-bundle-branch-block; PreD: Baloon pre-dilation; PostD: Baloon post-dilation.

### Calcification and Paravalvular Leakage

The 18 region maps for PVL vs the control group were analyzed in terms of volume and average intensity in FIGS. 11a, e, f; FIGS. 12a, b, c, d, e; FIGS. 14a, b. The calcific deposition was observed to be greater near the belly regions of the leaflets closer to annular center (R4-R9), while calcification near the root attachment area was greater in the control group and farther away from the annular center (R10-R18). In the control group, calcification patterns were more symmetric, compared with the PVL patients having greater absolute difference between adjacent regions. In terms of relative density, PVL patients had lower average calcific intensity compared to control (764[652-796] vs 801[730-856] Mean HU). Finally, Binomial logistic regression showed excellent classification power for a combined 36 parameter model using 18+18 volume and average intensity in each region (R2=0.56, p<0.01; cut-off=0.25, AUC=0.92, sensitivity=84.4%, specificity=85.2%).

### Calcification and Left Bundle Branch Block

The 18 region maps for LBBB vs the control group were analyzed in terms of volume and average intensity and the results are presented in FIGS. 11b, e, f; FIGS. 12f, g, h, i, j; FIGS. 14c, d. The calcific deposition was observed to be greater for most regions in the landing zone, particularly closer to the annular center (R1-R9). Furthermore, in the control group, calcification patterns were more symmetric, with LBBB patients having greater absolute difference between adjacent regions for all leaflets. In terms of relative density, LBBB patients had average calcific intensity compared to control (793[740-849] vs 801[730-856] Mean HU). Finally, Binomial logistic regression showed excellent classification power for a combined 30 parameter model using a 15+15 volume and average intensity map in each region (R2=0.612,p<0.05; cut-off=0.1, AUC=0.95, sensitivity=82%, specificity=78.6%)

### Calcification and Pre-Dilation

The 18 region maps for PreD vs the control group were analyzed in terms of volume and average intensity and the results are presented in FIGS. 11c, e, f; FIGS. 13a, b, c, d, e; FIGS. 14e, f. The calcific deposition was observed to be much higher across all landing zone regions, especially towards the non-coronary cusp. Furthermore, in the control group, calcification patterns were more symmetric, with PreD patients having greater absolute difference between adjacent regions, particularly between the leaflets. In terms of relative density, PreD patients had average calcific intensity compared to control (785[732-846] vs 801[730-856] Mean HU). Finally, Binomial logistic regression showed excellent classification power for a combined 36 parameter model using an 18+18 volume and average intensity map (R2=0.615, p=0.16; cut-off=0.1, AUC=0.95, sensitivity=84%, specificity=85%). Calcification and post-dilation

The 18 region maps for PostD vs the control group were analyzed in terms of volume and average intensity and the results are presented in FIGS. 11d, e, f; FIGS. 13f, g, h, i, j; FIGS. 14g, h. The calcific deposition was observed to be lower compared to the control group, with relatively lower deposition in the right and left coronary cusps. Furthermore, in the control group, calcification patterns were more symmetric, with PostD patients the asymmetry is most prominent between the right and left coronary cusps. In terms of relative density, PostD patients had higher average calcific intensity compared to control (839[712-916] vs 801[730-856] Mean HU). Finally, binomial logistic regression showed excellent classification power for a combined 36 parameter model using an 18+18 volume and average intensity map (R2=0.568, p<0.05; cut-off=0.22, AUC=0.916, sensitivity=86%, specificity=84%).

### DISCUSSION

The Improved TAVR valve designs, increased operator experience, and periprocedural planning with multimodality imaging has greatly reduced risks associated with TAVR [76, 77]. Accurate annular sizing is a robust way to reduce the risk of PVL and conduction abnormalities, and preprocedural sizing has been shown to reduce risks of mortality and morbidity [13, 22, 66]. Similarly, determination of the calcium burden and distribution pattern in the AV and left-ventricular outflow tract (LVOT) are predictors for PVL, annular rupture and the need for a second valve implantation [71, 78]. Contrast computed tomography angiography (CCTA) has become the gold standard for annular sizing and procedural planning for TAVR. CCTA offers a superior spatial and contrast resolution compared with 3D echocardiography and non-contrast CT for calcification quantification. Despite these advantages, current determination of the calcification burden relies exclusively on qualitative criteria to assess calcific severity [12]. Two key challenges are presented in quantification of calcific distribution using CCTA.

Various clinical studies investigated simpler modifications to the Agatston technique when using contrast-enhanced CT images. These approaches were mainly motivated by the idea of using a new cut-of thresholding value either fixed such as 450, 650 and 850 HU or dynamically determined using luminal attenuation added to some constant [17-20, 22]. Furthermore, techniques based on luminal attenuation, although reproducible, cannot be said to be precise, especially if there is image noise or calcific deposits in the region of interest.

The relationship between post-TAVR complications and TAVR risk assessment both with or without pre- or post-dilatation is ambiguous [22, 68, 79-81]. For example, the presence of valvular calcium is a mechanical barrier to the ideal device expansion, and it might seem appropriate to perform a pre-dilation to remove them. However, this approach can also cause stop non-circular valve expansion. Another point of contention is that while some support post-dilatation to prevent complications immediately following the deployment of the device, this may cause prosthetic leaflet damage which contributes to valve degeneration [82]. It appears that selecting the best course of action can be more confidently accomplished by carefully examining the unique 3D morphology and density distribution of the patient's valve calcification. The mechanical interaction between the deforming stent under the balloon pressure and dense calcium deposits anchored around the device landing zone may interfere with optimal final configuration [62, 83]. Even though the total calcification volume is well-studied parameter [13, 20, 22, 44], it is insufficient to predict how the calcification will interact with the stent without taking the location and relative density into account.

### Landing Zone Calcium Deposit Density and Location Proportionate to the Risk of PVL

It was demonstrated that patients diagnosed with PVL following TAVR had calcific depositions closer to the annular center with a lower relative density compared to those without PVL. Despite, having similar overall volumes to the control cases, the calcific lesions are flexed towards the annular center and have an arc-like shape with less calcification towards the root attachment. This pattern combined with a lower relative density may reduce the anchoring effect of the device upon deployment and increase the possibility of less sealing between the device skirt and the aortic annulus. The presence of this pattern had a very strong classification power to differentiation risk of PVL from the control. Therefore, this pattern can be used as a guide to determine valve size, degree of expansion, and the need for pre- or post-dilation.

### Landing Zone Calcium Deposit Density and Location Proportionate to the Risk of LBBB

Patients with LBBB following TAVR had much higher calcific deposition across most landing zone regions, with inter-leaflet asymmetry particularly between the left and non-coronary cusps. In addition, much higher calcific deposition was observed in the non-coronary cusp combined with a higher relative density compared to control. The regional volume and average intensity LBBB model pattern had excellent predictive power. Since the non-coronary cusp is in close proximity to the atrioventricular conduction system, stent expansion may push the calcification towards this region during deployment. As LBBB is one the most common risk factors associated with TAVR and may require permanent pace-maker implantation post TAVR, an ability to recognize patients with higher risk of developing this burdensome complication may help the treating physician in terms implantation height and the percentage of oversizing.

### Regional Volume and Density of Landing Zone Calcium can Indicate the Need for Pre or Post Dilation

Pre-dilatation and post-dilatation are both important interventional that accompany the TAVR. Both techniques aim to reduce the risk of peri-procedural complications, while also providing the maximal area of blood flow. Cost versus benefit analysis of both techniques is challenging due to lack of standardized quantifiable parameters for calcific burden. In that respect, the proposed calcification volume/density mapping scheme may prove as critical factors that can clearly stratify risk factors associated with both techniques. The described analysis showed that the necessity for pre- or post-dilatation is a function of both the relative density and distribution of the calcifications pattern with pre-dilation patterns implying a highly stenotic valve that may restricted the device expansion. Alternatively, patients who had post-dilation seem to have patterns that are much closer to control, with the exception of the left-coronary cusp calcification. This pattern indicates a high inter-leaflet calcific asymmetry which may provide a predictor for paravalvular leakage near the left-coronary and hence an important prognostic indicator for pre-operative planning of post-dilation and follow-up after intervention to assess possible device misconfiguration.

### Application of Post CT Images in Post-TAVR Clinical Assessment

While the use of CCTA pre-TAVR for planning and risk stratification is widely used, the role of CT imaging post-TAVR is not yet clear. Usually, post-TAVR CCTA is utilized when major complications, such as coronary occlusion, aortic breakdown, or bleeding, are suspected, and the role of routine post-TAVR CCTA is controversial [84]. Transesophageal echocardiography (TEE) is the imaging modality of choice post-TAVR to assess for residual gradients or any leaflet abnormalities, but the examination can be limited due to artifacts and acoustic shadowing as well as the invasive nature of TEE. CCTA has been shown to have increased sensitivity, compared to TEE, for detecting thrombi post-TAVR44. Additionally, MDCT can be used to assess the expansion, eccentricity and position of the valve, which relate to the PVL risk [12, 86]. The utility of determining the calcification patterns post-TAVR has yet to be fully investigated, and could potentially provide useful information about valve degeneration, risks for progression of calcifications or causes of PVL. The disclosed method was assessed on a sub-cohort of 37 patients to determine landing-zone calcific displacement and quantity after intervention. An association between device eccentricity and deformation with surrounding calcification of the native leaflet was found. This early result may provide a role for post-TAVR CT calcification assessment to diagnose new prosthetic valve diseases and help evaluate possible long-term damage to the implanted device.

### Conclusions from the Example Results

With increasing the usage of TAVR for AS treatment, there is little discussion beyond the total calcium volume score. Despite the evidence for the role of calcification patterns in prognosing different outcomes of TAVR, the role of relative calcific density and it's causal effect on valve expansion is limited. Described herein is a computational tool designed for detailed quantification of landing zone calcific location, quantity and relative density using routinely used CCTA imaging prescribed for TAVR planning. These new markers could be particularly important, as an add-on to exiting CT-TAVR guidance protocols given that in current CT-TAVR assessment, calcification severity is already confirmed, and valvular calcification is described qualitatively to plan the procedure [12]. The described computational tool for calcification burden in the aortic valve using CT images can be potentially used as: (1) A complementary method to existing TAVR workups, providing a way to differentiate qualitative degrees of landing zone calcification severity, which may lead to better evaluations of procedural techniques (Pre and post-dilation), optimal device type and sizing. (2) A tool for predicting patients with risk of PVL and LBBB (3) A way to detect and measure post-intervention calcific configuration which may prove as a useful diagnostic indicator for prosthetic valve related diseases. In one or more embodiments, predicting the probability of a periprocedural outcome/event may include a deep learning classification operation using a pretrained deep learning model based on the generated landing zone calcification model and patient characteristics.

In one or more embodiments, the methods described herein may be performed by executing instructions on computer readable media using a computer processor. Accordingly, in one embodiment there is provided a non-transitory computer readable medium comprising computer-executable instructions for assessing calcification of an aortic valve.

The non-transitory computer readable medium may be stored a local or remote hard disk or hard drive (of any type, including electromechanical magnetic disks and solid-state disks), a memory chip, including, e.g., random-access memory (RAM) and/or read-only memory (ROM), cache(s), buffer(s), flash memory, optical memory such as CD(s) and DVD(s), floppy disks, and any other form of storage medium in or on which information may be stored for any duration.

Different implementations of the disclosed method(s) may involve performing some or all the steps described herein in different orders or some or all of the steps substantially in parallel. Different implementations may involve performing some or all of the steps on different processors or the same processor, optionally wherein the processors are in networked communication. The functions or method steps may be implemented in a variety of programming languages known in the art. For example, such code or computer readable or executable instructions may be stored or adapted for storage in one or more machine-readable media, such as described above, which may be accessed by a processor-based system to execute the stored code or computer readable or executable instructions.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

[1] Hoffmann U, Massaro J M, D'Agostino R B, Kathiresan S, Fox CS, O'Donnell C J. Cardiovascular Event Prediction and Risk Reclassification by Coronary, Aortic, and Valvular Calcification in the Framingham Heart Study. Journal of the American Heart Association. 2016; 5(2):1-11. doi:10.1161/JAHA.115.003144
[2] Rogers M A, Aikawa E. Cardiovascular calcification: artificial intelligence and big data accelerate mechanistic discovery. Nature Reviews Cardiology. 2019; 16(5):261-274. doi:10.1038/s41569-018-0123-8
[3] Wang Y, Osborne M T, Tung B, Li M, Li Y. Imaging Cardiovascular Calcification. Vol 7. American Heart Association Inc.; 2018. doi:10.1161/JAHA.118.008564
[4] Greenland P, Blaha M J, Budoff M J, Erbel R, Watson K E. Coronary Calcium Score and Cardiovascular Risk. Journal of the American College of Cardiology. 2018; 72(4):434-447. doi:10.1016/j.jacc.2018.05.027
[5] Nakahara T, Dweck M R, Narula N, Pisapia D, Narula J, Strauss H W. Coronary Artery Calcification: From Mechanism to Molecular Imaging. JACC. Cardiovascular Imaging. 2017; 10(5):582-593. doi: 10.1016/j jcmg.2017.03.005
[6] Genereux P, Stone G W, O'Gara P T, et al. Natural History, Diagnostic Approaches, and Therapeutic Strategies for Patients with Asymptomatic Severe Aortic Stenosis. Journal of the American College of Cardiology. 2016; 67(19):2263-2288. doi:10.1016/j.jacc.2016.02.057
[7] Izquierdo-Gomez M M, Hernandez-Betancor I, Garcia-Niebla J, Mari-Lopez B, Laynez-Cerdefta I, Lacalzada-Almeida J. Valve calcification in aortic stenosis: Etiology and diagnostic imaging techniques. BioMed Research International. 2017;2017. doi:10.1155/2017/5178631
[8] Clavel M A, Pibarot P. A Decade of Revolutions in Calcific Aortic Steno-sis. Vol 38. W.B. Saunders; 2020. doi:10.1016/j.ccl.2019.10.001
[9] Lindman B R, Clavel M A, Mathieu P, et al. Calcific aortic stenosis. Na-ture Reviews Disease Primers. 2016; 2(1):1-28. doi:10.1038/nrdp.2016.6
[10] Siontis G C M, Overtchouk P, Cahill T J, et al. Transcatheter aortic valve implantation vs. surgical aortic valve replacement for treatment of symptomatic severe aortic stenosis: an updated meta-analysis. European Heart Journal. 2019; 40(38):3143-3153. doi:10.1093/eurheartj/ehz275
[11] Pollari F, Hitzl W, Vogt F, et al. Aortic valve calcification as a risk factor for major complications and reduced survival after transcatheter re-placement. Journal of Cardiovascular Computed Tomography. 2020; 14(4):307-313. doi:10.1016/j.jcct.2019.12.001
[12] Blanke P, Weir-McCall J R, Achenbach S, et al. Computed tomography imaging in the context of transcatheter aortic valve implantation (TAVI)/transcatheter aortic valve replacement (TAVR): An expert consensus document of the Society of Cardiovascular Computed Tomography. Journal of Cardiovascular Computed Tomography. 2019; 13(1):1-20. doi:10.1016/j.jcct.2018.11.008
[13] Khalique O K, Hahn R T, Gada H, et al. Quantity and Location of Aortic Valve Complex Calcification Predicts Severity and Location of Paravalvular Regurgitation and Frequency of Post-Dilation After Balloon-Expandable Transcatheter Aortic Valve Replacement. JACC: Cardiovascular Interventions. 2014; 7(8):885-894. doi:10.1016/j.jcin.2014.03.007
[14] Genereux P, Head S J, Hahn R, et al. Paravalvular Leak after Transcatheter Aortic Valve Replacement: The New Achilles' Heel?A Comprehensive Review of the Literature. Vol 61. Elsevier USA; 2013. doi:10.1016/j.jacc.2012.08.1039
[15] Pollari F, Dell'Aquila A M, Sohn C, et al. Risk factors for paravalvular leak after transcatheter aortic valve replacement. Journal of Thoracic and Cardiovascular Surgery. 2019; 157(4):1406-1415.e3. doi:10.1016/j.jtcvs.2018.08.085
[16] Mauri V, Deuschl F, Frohn T, et al. Predictors of paravalvular regurgitation and permanent pacemaker implantation after TAVR with a next-generation self-expanding device. Clinical Research in Cardiology. 2018; 107(8):688-697. doi:10.1007/s00392-018-1235-1
[17] Alqahtani A M, Boczar K E, Kansal V, Chan K, Dwivedi G, Chow B J W. Quantifying Aortic Valve Calcification using Coronary Computed Tomography Angiography. J Cardiovasc Comput Tomogr. 2017; 11(2):99-104. doi:10.1016/j.jcct.2017.01.007
[18] Cartlidge T R, Bing R, Kwiecinski J, et al. Contrast-enhanced computed tomography assessment of aortic stenosis. Heart. 2021;0:heartjnl-2020-318556. doi:10.1136/heartjnl-2020-318556
[19] Eberhard M, Mastalerz M, Frauenfelder T, et al. Quantification of aortic valve calcification on contrast-enhanced CT of patients prior to transcatheter aortic valve implantation. Eurolntervention. 2017; 13(8):921-927. doi:10.4244/EIJ-D-17-00060
[20] Bettinger N, Khalique O K, Krepp J M, et al. Practical determination of aortic valve calcium volume score on contrast-enhanced computed tomography prior to transcatheter aortic valve replacement and impact on paravalvular regurgitation: Elucidating optimal threshold cutoffs. J Cardiovasc Comput Tomogr. 2017; 11(4):302-308. doi:10.1016/j.jcct.2017.04.009
[21] Kim W K, Renker M, Rolf A, et al. Accuracy of device landing zone calcium volume measurement with contrast-enhanced multidetector computed tomography. International Journal of Cardiology. 2018; 263:171-176. doi:10.1016/j.ijcard.2018.02.042
[22] Jilaihawi H, Makkar R R, Kashif M, et al. A revised methodology for aortic-valvar complex calcium quantification for transcatheter aortic valve implantation. Eur Heart J Cardiovasc Imaging. 2014; 15(12):1324-1332. doi:10.1093/ehjci/jeu162
[23] Pawade T, Clavel M A, Tribouilloy C, et al. Computed Tomography Aortic Valve Calcium Scoring in Patients with Aortic Stenosis. Circulation: Cardiovascular Imaging. 2018;11(3). doi:10.1161/CIRCIMAGING.117.007146
[24] Baumgartner H, Hung J, Bermejo J, et al. Recommendations on the Echocardiographic Assessment of Aortic Valve Stenosis: A Focused Update from the European Association of Cardiovascular Imaging and the American Society of Echocardiography. Journal of the American Society of Echocardiography. 2017; 30(4):372-392. doi:10.1016/j.echo.2017.02.009
[25] Ahrens J, Geveci B, Law C. ParaView: An End-User Tool for Large-Data Visualization. In: The Visualization Handbook.; 2005. doi:10.1016/B978-012387582-2/50038-1
[26] Johnson H J, McCormick M M. The ITK Software Guide Book 2: Design and Functionality Fourth Edition.:552.
[27] McCormick M M, Liu X, Ibanez L, Jomier J, Marion C. ITK: enabling reproducible re-search and open science. Front Neuroinform. 2014;8. doi:10.3389/fninf.2014.00013
[28] Visual Computing for Medicine. Elsevier; 2014. doi:10.1016/C2011-O-05785-X
[29] Johnson H J, McCormick M M, Ibanez L. The ITK Software Guide Book 2: Design and Functionality—Volume 2. Kitware, Inc.; 2015.
[30] Schroeder W, Martin K, Lorensen B. The Visualization Toolkit: An Object-Oriented Approach to 3D Graphics; [Visualize Data in 3D—Medical, Engineering or Scientific; Build Your Own Applications with C++, Tcl, Java or Python; Includes Source Code for VTK (Supports Unix, Windows and Mac). 4. ed. Kitware, Inc; 2006.
[31] Lehmann G. Label object representation and manipulation with ITK. The Insight Journal. Published online Aug. 1, 2007. doi:10.54294/q6auw4
[32] Pawade T, Sheth T, Guzzetti E, Dweck M R, Clavel M A. Why and How to Measure Aortic Valve Calcification in Patients With Aortic Stenosis. JACC: Cardiovascular Imaging. 2019; 12(9):1835-1848. doi:10.1016/j.jcmg.2019.01.045
[33] Tustison N J, Gee J. Introducing Dice, Jaccard, and Other Label Overlap Measures To ITK. The Insight Journal. Published online Dec. 3, 2009. doi:10.54294/1vixgg
[34] Piazza N, de Jaegere P, Schultz C, Becker A E, Serruys P W, Anderson R H. Anatomy of the aortic valvar complex and its implications for transcatheter implantation of the aortic valve. Circ Cardiovasc Interv. 2008; 1(1):74-81. doi:10.1161/CIRCINTERVENTIONS.108.780858
[35] Clavel M A, Messika-Zeitoun D, Pibarot P, et al. The complex nature of discordant severe calcified aortic valve disease grading: new insights from combined Doppler echocardiographic and computed tomographic study. J Am Coll Cardiol. 2013; 62(24):2329-2338. doi:10.1016/j.jacc.2013.08.1621
[36] Agatston A S, Janowitz W R, Hildner F J, Zusmer N R, Viamonte M, De-trano R. Quantification of coronary artery calcium using ultrafast computed tomography. Journal of the American College of Cardiology. 1990; 15(4):827-832. doi:10.1016/0735-1097(90)90282-T
[37] Thubrikar M J, Aouad J, Nolan S P. Patterns of calcific deposits in operatively excised stenotic or purely regurgitant aortic valves and their relation to mechanical stress. The American Journal of Cardiology. 1986; 58(3):304-308. doi:10.1016/0002-9149(86)90067-6
[38] Back M, Gasser T C, Michel J B, Caligiuri G. Biomechanical factors in the biology of aortic wall and aortic valve diseases. Cardiovascular Re-search. 2013; 99(2):232-241. doi:10.1093/cvr/cvt040
[39] Arzani A, Mofrad M R K. A strain-based finite element model for calcification progression in aortic valves. Journal of Biomechanics. 2017; 65:216-220. doi:10.1016/j.jbiomech.2017.10.014
[40] Halevi R, Hamdan A, Marom G, Mega M, Raanani E, Haj-Ali R. Progressive aortic valve calcification: Three-dimensional visualization and biomechanical analysis. Journal of Biomechanics. 2015; 48(3):489-497. doi:10.1016/j.jbiomech.2014.12.004
[41] Myasoedova V A, Di Minno A, Songia P, et al. Sex-Specific Differences in Age-Related Aortic Valve Calcium Load: A Systematic Review and Meta-Analysis. Vol 61. Elsevier Ireland Ltd; 2020. doi:10.1016/j.arr.2020.101077
[42] Thaden J J, Nkomo V T, Suri R M, et al. Sex-related differences in calcific aortic stenosis: correlating clinical and echocardiographic characteristics and computed tomography aortic valve calcium score to excised aortic valve weight. European Heart Journal. 2016; 37(8):693-699. doi:10.1093/eurheartj/ehv560
[43] Blaha M J, Mortensen M B, Kianoush S, Tota-Maharaj R, Cainzos-Achirica M. Coronary Artery Calcium Scoring: Is It Time for a Change in Methodology?JACC: Cardiovascular Imaging. 2017; 10(8):923-937. doi:10.1016/j.jcmg.2017.05.007
[44] Hansson N C, Norgaard B L, Barbanti M, et al. The impact of calcium volume and distribution in aortic root injury related to balloon-expandable transcatheter aortic valve replacement. J Cardiovasc Comput Tomogr. 2015; 9(5):382-392. doi:10.1016/j.jcct.2015.04.002
[45] Angelillis M, Costa G, De Backer O, et al. Threshold for calcium volume evaluation in patients with aortic valve stenosis: correlation with Agatston score. J Cardiovasc Med (Hagerstown). Published online Dec. 3, 2020. doi:10.2459/JCM.0000000000001 145
[46] van der Bijl N, de Bruin P W, Geleijns J, et al. Assessment of coronary artery calcium by using volumetric 320-row multi-detector computed tomography: comparison of 0.5 mm with 3.0 mm slice reconstructions. Int J Cardiovasc Imaging. 2010; 26(4):473-482. doi:10.1007/s10554-010-9581-8
[47] Kalisz K, Buethe J, Saboo S S, Abbara S, Halliburton S, Rajiah P. Artifacts at Cardiac C T: Physics and Solutions. RadioGraphics. 2016; 36(7):2064-2083. doi:10.1148/rg.2016160079
[48] Sprem J, Vos B D de, Lessmann N, et al. Coronary calcium scoring with partial volume correction in anthropomorphic thorax phantom and screening chest CT images. PLOS ONE. 2018;13(12):e0209318. doi:10.1371/journal.pone.0209318
[49] Guzzetti E, Simard L, Clisson M, Clavel M A. Multiplanar “En Face” Reconstruction of the Aortic Valve. JACC: Cardiovascular Imaging. 2020; 13(12):2678-2680. doi:10.1016/j.jcmg.2020.05.021
[50] Piazza N, de Jaegere P, Schultz C, Becker A E, Serruys P W, Anderson R H. Anatomy of the aortic valvar complex and its implications for transcatheter implantation of the aortic valve. Circ Cardiovasc Interv. 2008; 1(1):74-81. doi:10.1161/CIRCINTERVENTIONS.108.780858
[51] Schsfers H J, Schmied W, Marom G, Aicher D. Cusp height in aortic valves. The Journal of Thoracic and Cardiovascular Surgery. 2013; 146(2):269-274. doi:10.1016/j.jtcvs.2012.06.053
[52] Pawade T A, Newby D E, Dweck M R. Calcification in Aortic Stenosis: The Skeleton Key. Journal of the American College of Cardiology. 2015; 66(5):561-577. doi:10.1016/j.jacc.2015.05.066
[53] Genereux P, Pibarot P, Redfors B, et al. Staging classification of aortic stenosis based on the extent of cardiac damage. European Heart Journal. 2017; 38(45):3351-3358. doi:10.1093/eurheartj/ehx381
[54]. Carabello B A, Paulus W J. Aortic stenosis. The Lancet 2009; 373:956-966.
[55] Boskovski M T, Gleason T G. Current Therapeutic Options in Aortic Stenosis. Circulation Research 2021:1398-1417.
[56] Pawade T, Sheth T, Guzzetti E, Dweck M R, Clavel M-A. Why and How to Measure Aortic Valve Calcification in Patients With Aortic Stenosis. JACC: Cardiovascular Imaging 2019; 12:1835-1848.
[57] Ko E, Kang D-Y, Ahn J-M, Kim T O, Kim J H, Lee J, Lee S-A, Kim D-H, Kim H J, Kim J B, Choo S J, Park S-J, Park D-W. Association of aortic valvular complex calcification burden with procedural and long-term clinical outcomes after transcatheter aortic valve replacement. European Heart Journal—Cardiovascular Imaging 2022; 23:1502-1510.
[58] Guddeti R R, Gill G S, Garcia-Garcia H M, Alla V M. Transcatheter aortic valve replacement in mixed aortic valve disease: a systematic review and meta-analysis. European Heart Journal—Quality of Care and Clinical Outcomes 2022; 8:169-176.
[59] Young M N, Kearing S, Malenka D, Goodney P P, Skinner J, Iribarne A. Geographic and Demographic Variability in Transcatheter Aortic Valve Replacement Dispersion in the United States. Journal of the American Heart Association 2021;10:e019588.
[60] Nuis R-J, Belle E van, Teles R, Blackman D, Veulemans V, Santos I A, Pilgrim T, Tarantini G, Saia F, lakovou I, Mascherbauer J, Vincent F, Geleijnse M, Sathananthan J, Wood D, Makkar R, Van Mieghem N M. Balloon expandable vs. Self expanding transcatheter valve for degenerated bioprosthesis: design and rationale of the BASELINE trial. American Heart Journal 2023; 256:139-147.
[61] Corrigan F E, Gleason P T, Condado J F, Lisko J C, Chen J H, Kamioka N, Keegan P, Howell S, Clements S D, Babaliaros V C, Lerakis S. Imaging for Predicting, Detecting, and Managing Complications After Transcatheter Aortic Valve Replacement. JACC: Cardiovascular Imaging 2019; 12:904-920.
[62] Sturla F, Ronzoni M, Vitali M, Dimasi A, Vismara R, Preston-Maher G, Burriesci G, Votta E, Redaelli A. Impact of different aortic valve calcification patterns on the outcome of transcatheter aortic valve implantation: A finite element study. Journal of Biomechanics 2016; 49:2520-2530.
[63] Rheude T, Pellegrini C, Allali A, Bleiziffer S, Kim W-K, Neuser J, Landt M, Rudolph T, Renker M, Widder J D, Qu L, Alvarez-Covarrubias H A, Mayr N P, Richardt G, Xhepa E, Joner M. Multicenter comparison of latest-generation balloon-expandable versus self-expanding transcatheter heart valves: Ultra versus Evolut. International Journal of Cardiology 2022; 357:115-120.
[64] Kim W-K, Bhumimuang K, Renker M, Fischer-Rasokat U, Mollmann H, Walther T, Choi Y-H, Nef H, Hamm C W. Determinants of paravalvular leakage following transcatheter aortic valve replacement in patients with bicuspid and tricuspid aortic stenosis. European Heart Journal—Cardiovascular Imaging 2021; 22:1387-1396.
[65] Pio S M, Bax J, Delgado V. How valvular calcification can affect the outcomes of transcatheter aortic valve implantation. Expert Review of Medical Devices 2020; 17:773-784.
[66] Binder R K, Webb J G, Toggweiler S, Freeman M, Barbanti M, Willson A B, Alhassan D, Hague C J, Wood D A, Leipsic J. Impact of post-implant SAPIEN XT geometry and position on conduction disturbances, hemodynamic performance, and paravalvular regurgitation. JACC: Cardiovascular Interventions 2013; 6:462-468.
[67] Zaid S, Ahmad H, Kaple R, Undemir C, Lansman S L, Dangas G D, Sharma S K, Kini A, Tang G H L. Novel Predictors of New Persistent Left Bundle Branch Block After SAPIEN 3 Transcatheter Aortic Valve Replacement. JACC: Cardiovascular Interventions 2020; 13:1141-1143.
[68] McInerney A, Vera-Urquiza R, Tirado-Conte G, Marroquin L, Jimenez-Quevedo P, NuAez-Gil I, Pozo E, Gonzalo N, Agustin J A de, Escaned J, Fernendez-Ortiz A, Macaya C, Nombela-Franco L. Pre-dilation and Post-dilation in Transcatheter Aortic Valve Replacement: Indications, Benefits and Risks. Interv Cardiol 2021;16:e28.
[69] De Sciscio P, Brubert J, De Sciscio M, Serrani M, Stasiak J, Moggridge G D. Quantifying the Shift Toward Transcatheter Aortic Valve Replacement in Low-Risk Patients. Circulation: Cardiovascular Quality and Outcomes 2017;10:e003287.
[70] Morany A, Lavon K, Halevi R, Haj-Ali N, Bluestein D, Raanani E, Hamdan A, Haj-Ali R. Fragmentation of Different Calcification Growth Patterns in Bicuspid Valves During Balloon Valvuloplasty Procedure. Ann Biomed Eng 2022.
[71] Okuno T, Asami M, Heg D, Lanz J, Praz F, Hagemeyer D, Brugger N, Grani C, Huber A, Spirito A, Rsber L, Stortecky S, Windecker S, Pilgrim T. Impact of Left Ventricular Outflow Tract Calcification on Procedural Outcomes After Transcatheter Aortic Valve Replacement. JACC: Cardiovascular Interventions 2020; 13:1789-1799.
[72] Hwang I-C, Park J-B, Lee W, Han J-K, Kim C-H, Lee S-P, Yang H-M, Park E-A, Kim H-K, Kim Y-J, Koo B-K, Sohn D-W, Ahn H, Kim H-S. P3290Eccentricity of aortic valve calcification predicts risk of paravalvular regurgitation and response to balloon post-dilation after self-expandable transcatheter aortic valve replacement. European Heart Journal 2017;38:ehx504.P3290.
[73] Pajjuru V S, Thandra A, Guddeti R R, Walters R W, Jhand A, Andukuri V G, Alkhouli M, Spertus J A, Alla V M. Sex differences in mortality and 90-day readmission rates after transcatheter aortic valve replacement: a nationwide analysis from the USA. European Heart Journal—Quality of Care and Clinical Outcomes 2022; 8:135-142.
[74] Bak M, Choi K H, Kim J H, Park T K, Kim E K, Kim S M, Choi S H, Park S J. Risk factors and clinical effects of late leaflet thrombosis after transcatheter aortic valve
[75]G enereux P, Piazza N, Alu M C, Nazif T, Hahn R T, Pibarot P, Bax J J, Leipsic J A, Blanke P, Blackstone E H, Finn M T, Kapadia S, Linke A, Mack M J, Makkar R, Mehran R, Popma J J, Reardon M, Rodes-Cabau Josep, Van M N M, Webb J G, Cohen D J, Leon M B. Valve Academic Research Consortium 3: Updated Endpoint Definitions for Aortic Valve Clinical Research. Journal of the American College of Cardiology 2021; 77:2717-2746.
[76] Kanwar A, Thaden J J, Nkomo V T. Management of Patients With Aortic Valve Stenosis. Mayo Clinic Proceedings 2018; 93:488-508.
[77] Eggebrecht H, Vaquerizo B, Moris C, Bossone E, Lammer J, Czerny M, Zierer A, Schrofel H, Kim W K, Walther T, Scholtz S, Rudolph T, Hengstenberg C, Kempfert J, Spaziano M, Lefevre T, Bleiziffer S, Schofer J, Mehilli J, Seiffert M, Naber C, Biancari F, Eckner D, Cornet C, Lhermusier T, Philippart R, Siljander A, Giuseppe Cerillo A, Blackman D, Chieffo A, Kahlert P, Czerwinska-Jelonkiewicz K, Szymanski P, Landes U, Kornowski R, D′Onofrio A, Kaulfersch C, Sondergaard L, Mylotte D, Mehta R H, De Backer O. Incidence and outcomes of emergent cardiac surgery during transfemoral transcatheter aortic valve implantation (TAVI): Insights from the European Registry on emergent Cardiac Surgery during TAVI (EuRECS-TAVI). European Heart Journal 2018; 39:676-684.
[78] Rys M, Hryniewiecki T, Michakowska I, Stokkosa P, R6zewicz-Juraszek M, Chmielak Z, Dgbrowski M, Mirota K, Szymanski P. Quantitative estimation of aortic valve calcification in multislice computed tomography in predicting the development of paravalvular leaks following transcatheter aortic valve replacement. Advances in Interventional Cardiology 2018; 14:85-89.
[79] Butter C, Bramlage P, Rudolph T, Jacobshagen C, Rothe J, Treede H, Kerber S, Frank D, Seilerova L, Schymik G. Balloon expandable transcatheter aortic valve implantation via the transfemoral route with or without pre-dilation of the aortic valve rationale and design of a multicentre registry (EASE-IT TF). BMC Cardiovasc Disord 2016; 16:223.
[80] Grube E, Naber C, Abizaid A, Sousa E, Mendiz O, Lemos P, Kalil F R, Mangione J, Buellesfeld L. Feasibility of Transcatheter Aortic Valve Implantation Without Balloon Pre-Dilation. JACC: Cardiovascular Interventions 2011; 4:751-757.
[81] Finotello A, Morganti S, Auricchio F. Finite element analysis of TAVI: Impact of native aortic root computational modeling strategies on simulation outcomes. Medical Engineering & Physics 2017; 47:2-12.
[82] Kostyunin A E, Yuzhalin A E, Rezvova M A, Ovcharenko E A, Glushkova T V, Kutikhin A G. Degeneration of Bioprosthetic Heart Valves: Update 2020. JAHA 2020;9.
[83] Esmailie F, Razavi A, Yeats B, Sivakumar S K, Chen H, Samaee M, Shah I A, Veneziani A, Yadav P, Thourani V H, Dasi L P. Biomechanics of Transcatheter Aortic Valve Replacement Complications and Computational Predictive Modeling. Structural Heart 2022; 6:100032.
[84] Chiocchi M, Ricci F, Pasqualetto M, D′Errico F, Benelli L, Pugliese L, Cavallo A U, Forcina M, Presicce M, De Stasio V, Di Donna C, Di Tosto F, Spiritigliozzi L, Floris R, Romeo F. Role of computed tomography in transcatheter aortic valve implantation and valve-in-valve implantation: Complete review of preprocedural and postprocedural imaging. Journal of Cardiovascular Medicine 2020:21:182-191.
[85] Leetmaa T, Hansson N C, Leipsic J, Jensen K, Poulsen S H, Andersen H R, Jensen J M, Webb J, Blanke P, Tang M, Norgaard B L. Early Aortic Transcatheter Heart Valve Thrombosis: Diagnostic Value of Contrast-Enhanced Multidetector Computed Tomography. Circulation: Cardiovascular Interventions 2015; 8:1-8.
[86] Willson A B, Webb J G, Labounty T M, Achenbach S, Moss R, Wheeler M, Thompson C, Min J K, Gurvitch R, Norgaard B L, Hague C J, Toggweiler S, Binder R, Freeman M, Poulter R, Poulsen S, Wood D A, Leipsic J. 3-dimensional aortic annular assessment by multidetector computed tomography predicts moderate or severe paravalvular regurgitation after transcatheter aortic valve replacement: A multicenter retrospective analysis. Journal of the American College of Cardiology 2012; 59:1287-1294.

We claim:

1. A method for assessing calcification of an aortic valve, the method comprising:
receiving computed tomography (CT) images of the aortic valve;
pre-processing the received images to have a field of view focused on an aortic root corresponding to the aortic valve;
implementing a fast-marching method for the pre-processed images to segment the aortic valve from surrounding tissue to generate an aortic root model;
determining multiple principal axes and multiple landmark points based on the pre-processed images and the aortic root model to define a local coordinate system relative to leaflets of the aortic valve;
generating a calcification model based on the pre-processed images and the aortic root model by iteratively changing an initial estimate of calcific Hounsfield (HU) threshold until a minimum false positive rate (FPR) criterion is reached; and
generating an indicator quantifying calcification of the aortic valve based on the calcification model, the principal axes and the landmark points.

2. The method of claim 1, wherein generating the indicator includes generating one or more maps quantifying calcification of the aortic valve.

3. The method of claim 2 further comprising measuring one or more local and/or global geometric parameters quantifying calcification of the aortic valve based on the one or more maps.

4. The method of claim 3 wherein the geometric parameters include one or more of a physical volume, multiple principal orientation axes, a center of mass, multiple boundary points, a roundness, a flatness, an elongation, an equivalent spherical radius, equivalent ellipsoidal diameters and a fractal dimension index of each calcific lesion in the generated calcification model.

5. The method of claim 1, wherein the received images are pre-processed to have the field of view include an interface between the aortic valve and a left ventricular outflow tract (LVOT), and a part of the ascending aorta after a Sino-tubular junction (STJ); and to have the field of view exclude any other surrounding structures.

6. The method of claim 1, wherein generating the aortic root model includes performing morphological operations to obtain a smoothed surface.

7. The method of claim 1, wherein the multiple principal axes correspond to an anatomical short axis view, and two long axis views that are perpendicular to the “En-face” short axis view.

8. The method of claim 1 further comprising generating an anatomical N region volume map and/or an anatomical N region average intensity map quantifying calcification of the aortic valve, the anatomical N region volume map and the anatomical N region average intensity map being based on the calcification model, the principal axes, a Sino-tubular junction (STJ) height, an annular radius, angles between leaflets of the aortic valve, wherein N defines a number of discrete regions in the volume map and/or the average intensity map.

9. The method of claim 2, wherein the one or more maps quantifying calcification include one or more of a regional calcification map, a radial distance map, a longitudinal distance map and a calcification intensity map.

10. The method of claim 1, wherein the received CT images are contrast-enhanced CT images.

11. The method of claim 3, further comprising diagnosing, monitoring or prognosing aortic valve stenosis (AS) in a subject based on the one or more local and/or global geometric parameters.

12. The method of claim 11, wherein a procedural risk assessment and/or a complication/event prediction is conducted prior to a transcatheter aortic valve replacement (TAVR).

13. A system for assessing calcification of an aortic valve, the system comprising:
a processor; and
a memory storing processor-executable instructions, wherein the instruction configure the processor to perform the method of any of claims 1 to 12.
